(19) United States
(12) Patent Application Publication
(10) Pub. No.: US 2003/0144310 A1
(43) Pub. Date: Jul. 31, 2003

(54) Molecules specific for NPFF receptors and uses thereof

(76) Inventors: Lakmal W. Boteju, Cedar Grove, NJ (US); Michael J. Konkel, Garfield, NJ (US); Joel K. Kawakami, Newfoundland, NJ (US); John Wetzel, Fairlawn, NJ (US)

Correspondence Address: Cooper & Dunham LLP, 1185 Avenue of the Americas, New York, NY, 10036, US

(21) Appl. No.: 10/253,288

(22) Filed: Sep. 24, 2002

Related U.S. Application Data
(60) Provisional application No. 60/324,769, filed on Sep. 24, 2001.

(51) Int. Cl. A61K 31/505; A61K 31/445; A61K 31/4164; A61K 31/40; C07D 239/42; C07D 223/16; C07D 213/53; A61K 31/18

(52) U.S. Cl. 514/275; 514/317; 514/357; 514/408; 514/401; 514/605; 544/330; 546/229; 546/332; 548/354.1; 548/566; 564/95

(57) ABSTRACT

This invention provides a compound having the structure:

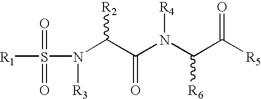

wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,

wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

wherein Y is O or S;

wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;

wherein R2 is —(CH₂)ₙ—NH—C(═NH)—NH₂; —(CH₂)n—C(═NH)—NH₂; —(CH₂)ₙ—N(R7)₂; or -J;

wherein n is an integer between 1 and 6, and wherein J is any of the following structures:

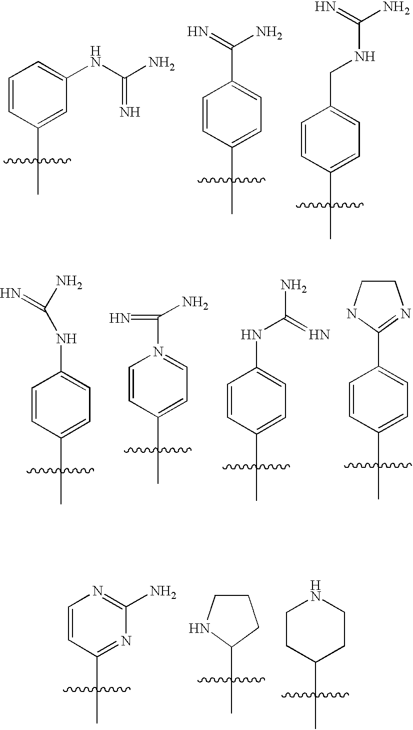

wherein each of R3 and R4 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; or C5-C7 cycloalkenyl;

wherein R5 is —OR8 or —N(R8)₂;

wherein each R8 is independently H, straight chained or branched C1-C7 alkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;

wherein R6 is arylalkyl, heteroarylalkyl, aryl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

or a pharmaceutically acceptable salt thereof. This invention also provides a method of treating a lower urinary tract disorder in a subject in need of such treatment comprising administering to the subject an effective amount of any of the aforementioned compounds.

Throughout this application, various publications are referenced within parentheses. Disclosures of these publications in their entireties are hereby incorporated by reference into this application to more fully describe the state of the art to which this invention pertains. Full bibliographic citations for these references may be found immediately preceding the claims.

### BACKGROUND OF THE INVENTION

Neuropeptide FF (NPFF), is an octapeptide isolated from bovine brain in 1985 by Yang (1). FMRFamide-like immmunoreactivity was observed in rat brain, spinal cord, and pituitary, suggesting the existence of mammalian homologs of the Phe-Met-Ag-Phe-amide (FMRFamide) family of invertebrate peptides. The isolation of NPFF, named for its N- and C-terminal phenylalanines and another mammalian peptide, NPAF, confirmed the existence of a mammalian family of peptides sharing the C-terminal homology with FMRFamide (1). NPFF is also called F8Famide and morphine modulating peptide, whereas NPAF is also called A18Famide in the literature. Molecular cloning has revealed that NPFF and NPAF are encoded from the same gene, and cleaved from a common precursor protein (2). Studies of the localization, radioligand binding, and function of NPFF-like peptides indicate they are neuromodulatory peptides whose effects are likely to be mediated by G protein-coupled receptors (See PCT International Publication No. WO 00/18438).

There are two known receptor subtypes for NPFF, NPFF-1 and NPFF-2 (3). Recently, two NPFF receptor subtypes (NPFF-1 and NPFF-2) were discovered and cloned from rat and human tissues. (4). The localization of protein and mRNA for these two receptors indicates that they may have utility as targets for drugs to treat a variety of disorders including, but not limited to, disorders of electrolyte balance, diabetes, respiratory disorders, gastrointestinal disorders, depression, phobias, anxiety, mood disorders, cognition/memory disorders, obesity, pain, alertness/sedation, lower urinary tract disorders and cardiovascular indications.

NPFF is an endogenous modulator of opioid systems with effects on morphine analgesia, tolerance, and withdrawal (5, 6). NPFF appears to represent an endogenous “anti-opioid” system in the CNS, acting at specific high-affinity receptors that are distinct from opioid receptors (7, 8). Endogenous NPFF has been suggested to play a role in morphine tolerance: agonists of NPFF precipitate “morphine abstinence syndrome” (symptoms of morphine withdrawal) in morphine-dependent animals (9, 10), while antagonists and anti-NPFF IgG restore morphine sensitivity and ameliorate symptoms of withdrawal. NPFF has also been shown to participate in the regulation of pain threshold, showing both “anti-opiate” effects and analgesic effects, depending on the test system (5).

The ability of NPFF peptides to modulate the opioid system raised the possibility that NPFF interacts directly with opiate receptors. However, radioligand binding assays using a tyrosine-substituted NPFF analog [¹²⁵I]Y8Fa demonstrate that NPFF acts through specific high affinity binding sites distinct from opiate receptors (11-14) that are sensitive to inhibition by guanine nucleotides (15).

NPFF and related peptidic agonists exhibit direct analgesic activity in some animal models. NPFF has been shown to produce analgesia in the rat tail-flick and paw pressure models, upon intrathecal administration (16). Similarly, a NPFF-like peptide, SLAAPQRF-amide, isolated from rat brain and spinal cord (17), produces antinociceptive action in the tail-flick and paw pressure models (18). NPFF has also been observed to play a role in animal models of chronic pain. For example, NPFF has recently been shown to be involved in inflammatory pain (19) and neuropathic pain (20). Importantly, NPFF was shown to attenuate the allodynia associated with neuropathic pain, suggesting that it may be clinically useful in treating this condition. NPFF also has been shown to produce nighttime hyperasthesic analgesia in the tail-flick test upon i.c.v. administration in the rat (21). A peptidic NPFF analog, (D)Tyr¹, (NMe)Phe³-NPFF (1DMe, 1DMeY8Fa), which is partially protected against enzymatic degradation and also has high affinity for its receptors, shows long-lasting analgesic activity in the above models upon intrathecal administration (22, 23). In carrageenan inflammation, 5-10 mmol of 1DMe was effective against both thermal hyperalgesia and mechanical allodynia, and in a neuropathic pain model, 1DMe showed antiallodynic effects against cold allodynia (24). 1DMe also shows analgesic activity in the rat vocalization threshold upon intrathecal administration (25).

Recent studies in our laboratories have shown that NPFF also has peripheral effects. NPFF and related agonists show decrease in the contraction frequency of the rat bladder upon i.v. and i.t. administration (See PCT International Publication No. WO 00/18438). A potent NPFF agonist, PFRF-amide, has been shown to increase blood pressure and heart rate in rats (26). In addition, NPFF and related peptides have a number of other biological activities that may be therapeutically relevant including effects on feeding (27-29), psychotic behavior (30), nicotine addiction (31), and other cardiovascular functions (32, 33).

Effects on feeding behavior are further supported by findings that demonstrate NPFF-like immunoreactive neurons, as well as NPFF1 receptor mRNA, localize to the hypothalamus (3,5). The NPFF1-selective ligand, BIBP 3226, which is also a neuropeptide Y Y1 antagonist, blocks feeding through a nonspecific mechanism, not secondary to inhibition of Y1 (38). These data suggest that feeding behavior may be regulated through a NPFF1 receptor mechanism.

It is thus evident that NPFF agonists and/or antagonists have great potential as being therapeutically useful agents for the treatment of a diverse array of clinically relevant human disorders. NPFF agonists may have therapeutic potential, among others, for the treatment of pain, memory loss, circadian rhythm disorders, and micturition disorders. Cloned receptor subtypes of NPFF and the development of high-efficiency in vitro assays, both for binding and receptor activation, have aided the discovery and development of novel NPFF ligands in our hands. Moreover, it is practically possible to design a molecule that is an agonist at one NPFF subtype, and an antagonist at the other(s). This concept of a dual-acting molecule provides an attractive means of designing drugs that can treat multiple disorders. These molecules may be used by themselves as drugs or as valuable tools for the design of drugs for the treatment of various clinical abnormalities in a subject wherein the abnormality is alleviated by increasing or decreasing the activity of a mammalian NPFF receptor by administering to the subject an amount of a compound which is an antagonist or agonist of mammalian NPFF receptors to effect a treatment of the abnormality.

Described herein are sulfonylamide-containing molecules, which act as agonists and/or antagonists at one or more NPFF receptor subtypes. Dansyl RFamide has been used by Brussaard (34) as an UV active pharmacological tool for studying the effects of FMRF amide and was later shown to bind to an NPFF receptor in rat tissue (7). A series of benzoyl-substituted dipeptides relating to RFamide has been described by Bourguignon et. al. (35). Described herein are unique sulfonamido-peptidomimetic ligands which are either antagonists and/or agonists that show selectivity towards NPFF receptor subtype(s). Also, described herein are unique compounds that have improved pharmacological properties at the NPFF receptor subtype.

### SUMMARY OF THE INVENTION

This invention provides a compound having the structure:

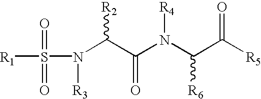

wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,

wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

wherein Y is O or S;

wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;

wherein R2 is —(CH₂)ₙ—NH—C(═NH)—NH₂; —(CH₂)n—C(═NH)—NH₂; —(CH₂)ₙ—N(R7)₂; or -J;

wherein n is an integer between 1 and 6, and wherein J is any of the following structures:

wherein each of R3 and R4 is independently H, straight chained

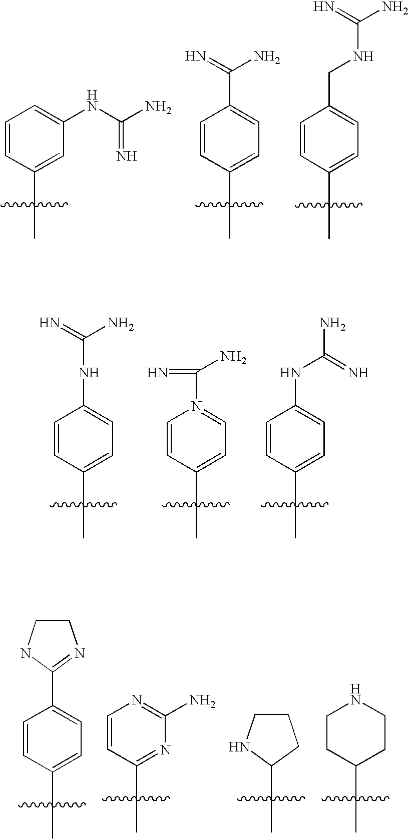

or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; or C5-C7 cycloalkenyl;

wherein R5 is —OR8 or —N(R8)₂;

wherein each R8 is independently H, straight chained or branched C1-C7 alkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;

wherein R6 is arylalkyl, heteroarylalkyl, aryl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

or a pharmaceutically acceptable salt thereof.

This invention also provides a method of treating pain is a subject in need of such treatment comprising administering to the subject an effective amount of the aforementioned compound.

This invention further provides a method of treating a lower urinary tract disorder in a subject in need of such treatment comprising administering to the subject an effective amount of the aforementioned compound.

### BRIEF DESCRIPTION OF THE FIGURES

FIG. 1: Shows the effect of compound (N-(4,7-dimethyl-2-quinolinyl)guanidine) on bladder activity in the anesthetized rat. Rhythmic elevations in bladder pressure, resulting from distension induced contractions, were unaffected by the i.v. administration of physiological saline. In contrast, the NPFF receptor ligand compound (N-(4,7-dimethyl-2-quinolinyl)guanidine) produced an immediate inhibition of bladder activity, which persisted for 12 min.

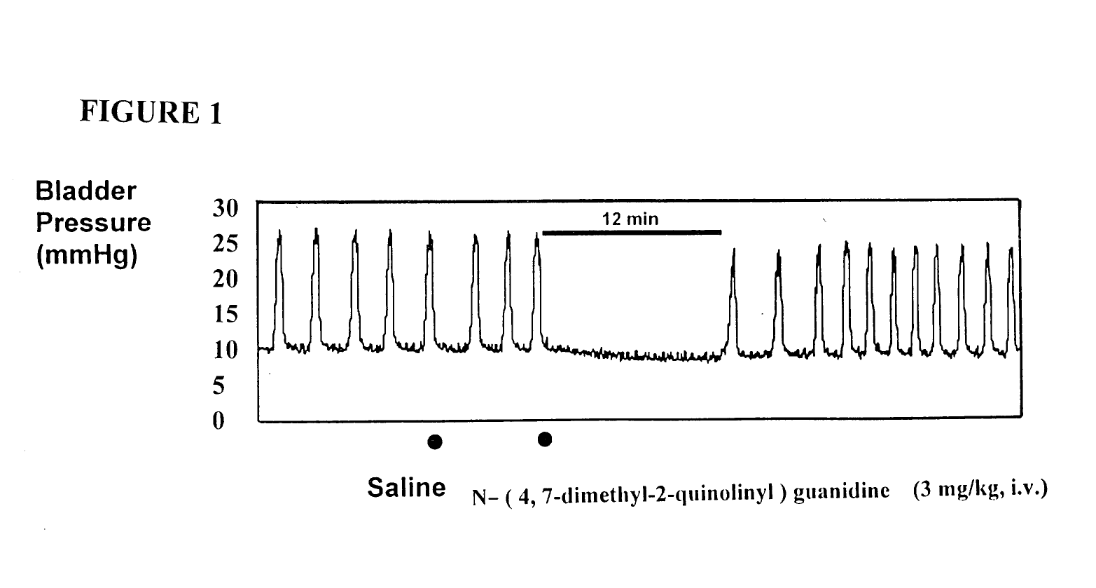

FIG. 2: Shows the effect of compound (N-(6-chloro-4-methyl-2-quinolinyl)guanidine) on bladder activity in the anesthetized rat. Rhythmic elevations in bladder pressure, resulting from distension induced contractions, were unaffected by the i.v. administration of physiological saline. In contrast, the NPFF receptor ligand compound (N-(6-chloro-4-methyl-2-quinolinyl)guanidine) produced an immediate inhibition of bladder activity, which persisted for 35 min.

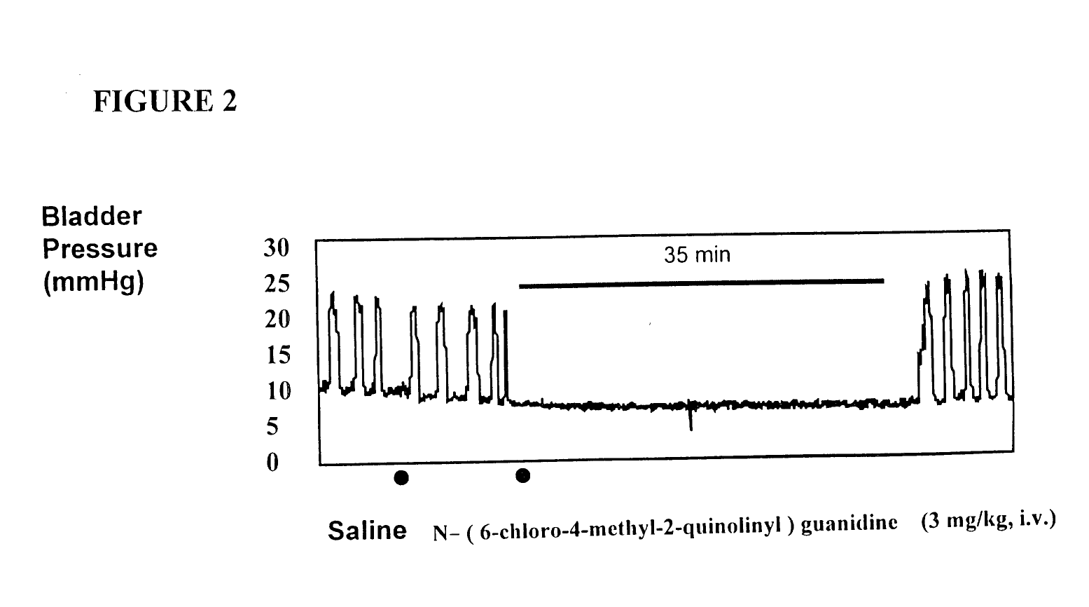

### DETAILED DESCRIPTION OF THE INVENTION

This invention provides a compound having the structure:

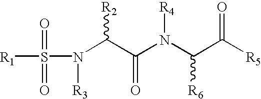

wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,

wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

wherein Y is O or S;

wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;

wherein R2 is —(CH₂)ₙ—NH—C(═NH)—NH₂; —(CH₂)n—C(═NH)—NH₂; —(CH₂)ₙ—N(R7)₂; or -J;

wherein n is an integer between 1 and 6, and wherein J is any of the following structures:

wherein each of R3 and R4 is independently H, straight chained

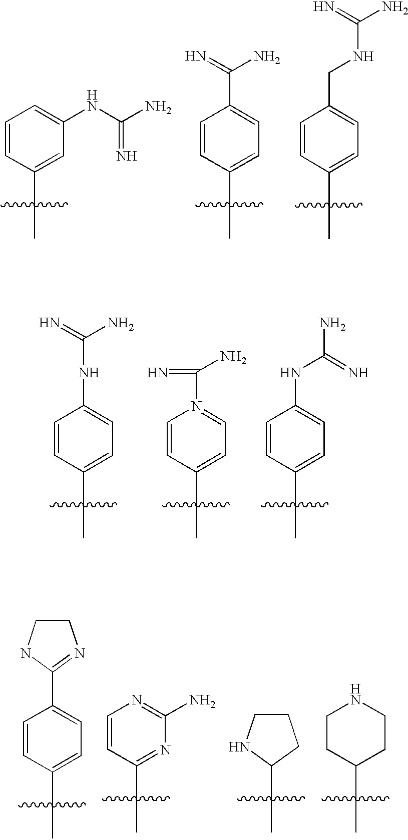

or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; or C5-C7 cycloalkenyl;

wherein R5 is —OR8 or —N(R8)₂;

wherein each R8 is independently H, straight chained or branched C1-C7 alkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;

wherein R6 is arylalkyl, heteroarylalkyl, aryl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

or a pharmaceutically acceptable salt thereof.

In one embodiment of the invention, when R1 is naphthyl, the naphthyl may be substituted with one or more of the following: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —C(═Y)NR7, —NR7C(═Y)R7 or —N(R7)C(═Y)N(R7)₂.

This invention also provides a compound having the structure:

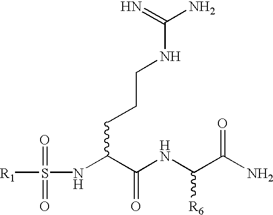

wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,

wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

wherein Y is O or S;

wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; and

wherein R6 is arylalkyl, heteroarylalkyl, aryl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,

or a pharmaceutically acceptable salt thereof.

This invention also provides a compound having the structure:

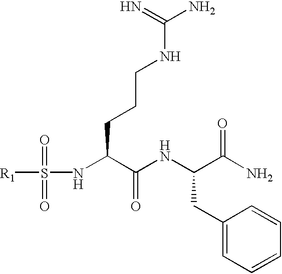

wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; or arylalkyl, heteroarylalkyl, phanyl or heteroaryl,

wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂.

In one embodiment, the compound has the structure:

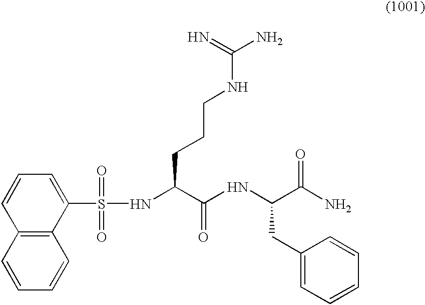

In another embodiment, the compound has the structure:

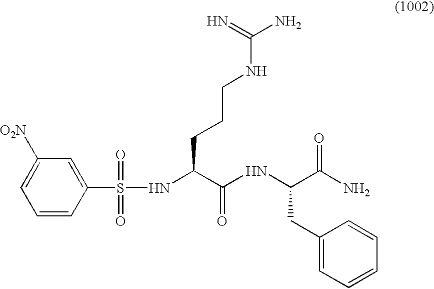

In another embodiment, the compound has the structure:

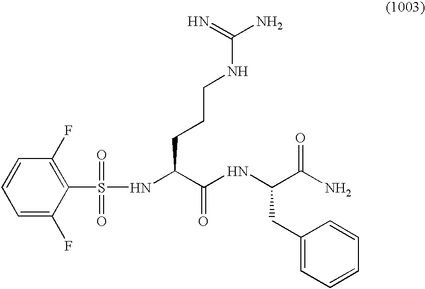

In another embodiment, the compound has the structure:

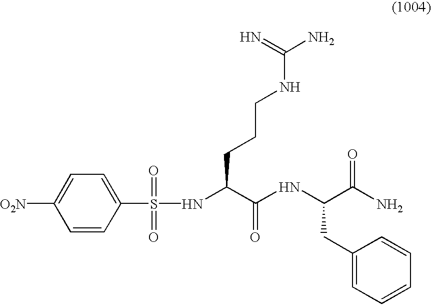

In another embodiment, the compound has the structure:

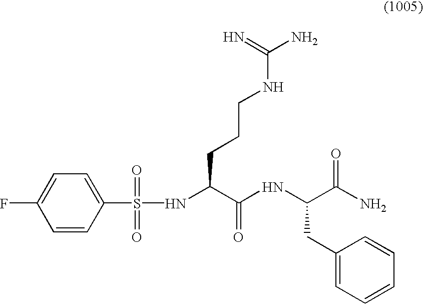

In another embodiment, the compound has the structure:

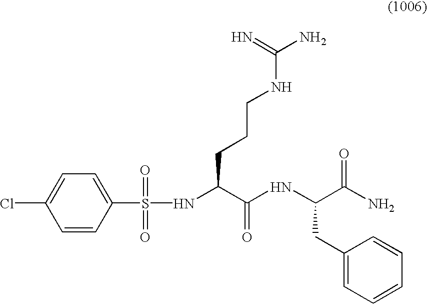

In another embodiment, the compound has the structure:

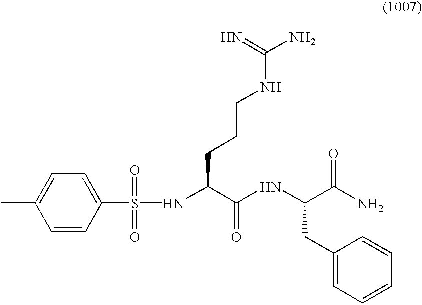

In another embodiment, the compound has the structure:

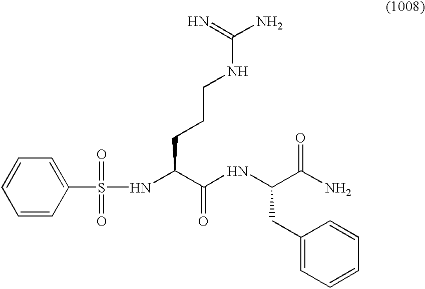

In another embodiment, the compound has the structure:

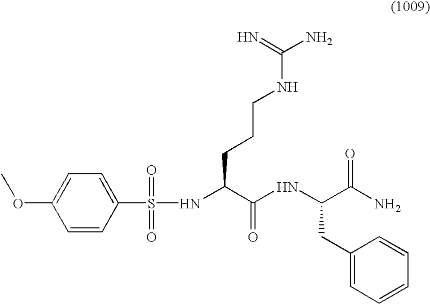

In another embodiment, the compound has the structure:

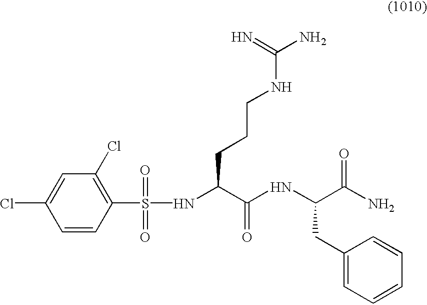

In another embodiment, the compound has the structure:

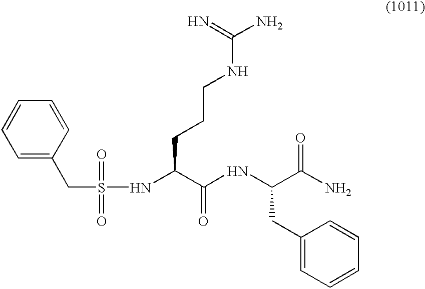

In another embodiment, the compound has the structure:

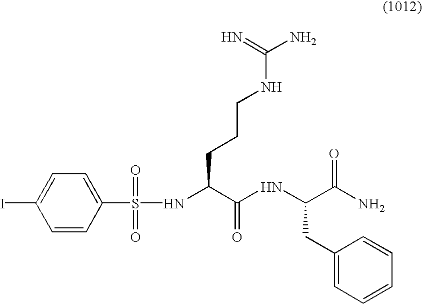

In another embodiment, the compound has the structure:

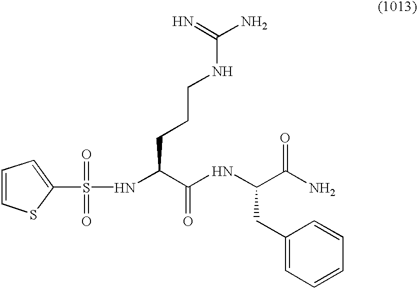

In another embodiment, the compound has the structure:

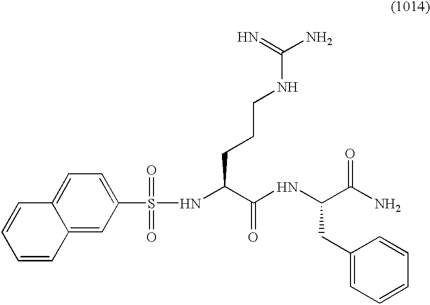

In another embodiment, the compound has the structure:

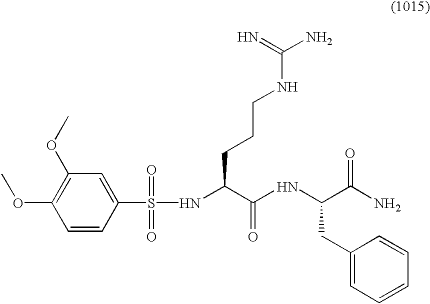

In another embodiment, the compound has the structure:

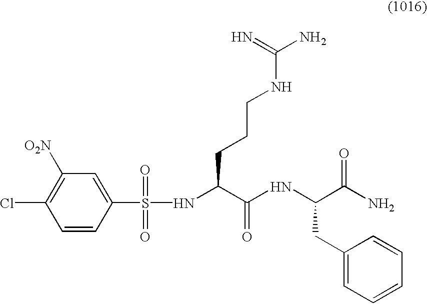

In another embodiment, the compound has the structure:

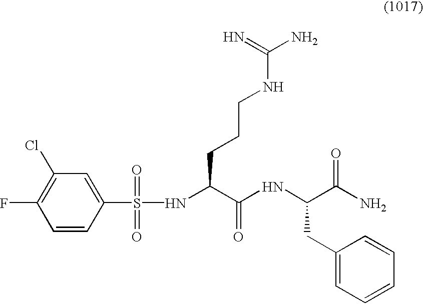

In another embodiment, the compound has the structure:

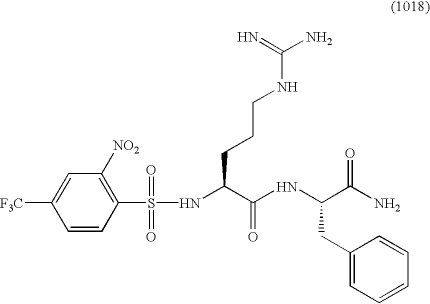

In another embodiment, the compound has the structure:

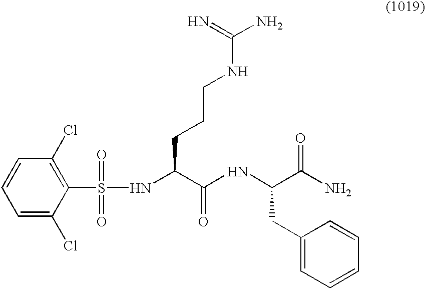

In another embodiment, the compound has the structure:

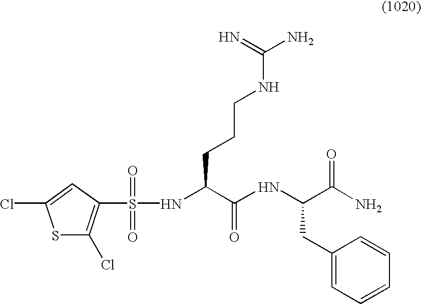

In another embodiment, the compound has the structure:

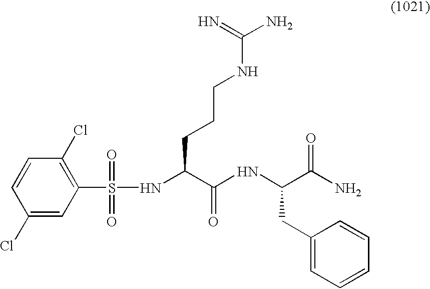

In another embodiment, the compound has the structure:

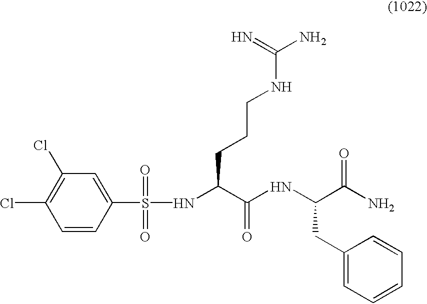

In another embodiment, the compound has the structure:

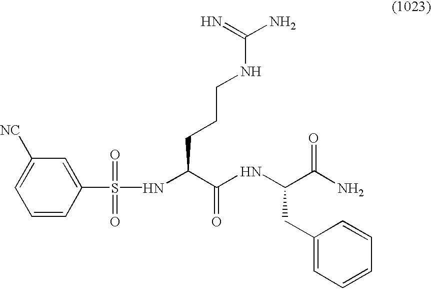

In another embodiment, the compound has the structure:

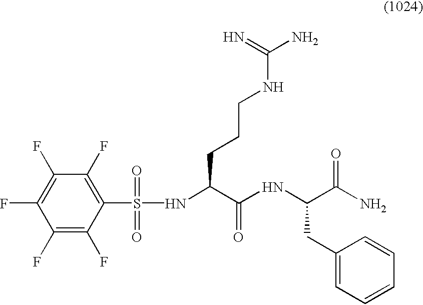

In another embodiment, the compound has the structure:

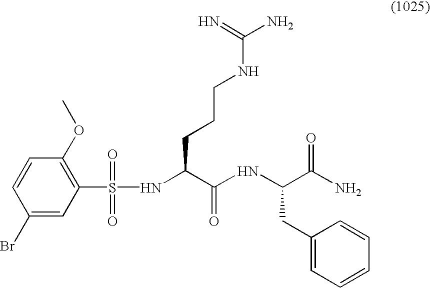

In another embodiment, the compound has the structure:

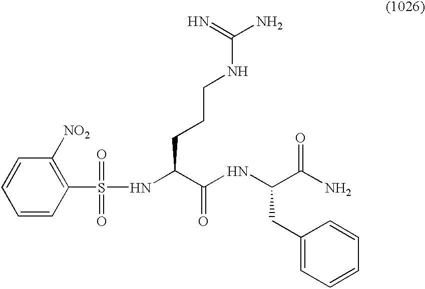

In another embodiment, the compound has the structure:

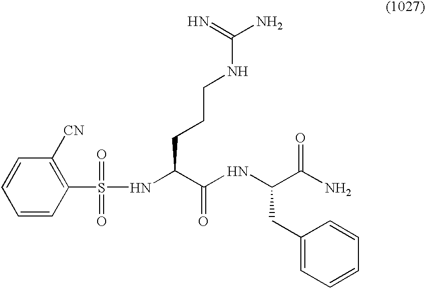

In another embodiment, the compound has the structure:

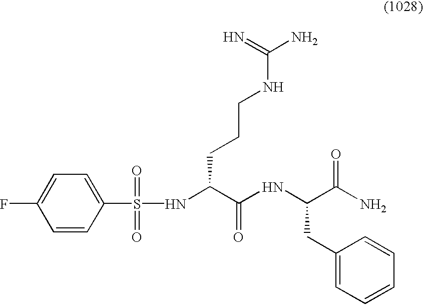

In another embodiment, the compound has the structure:

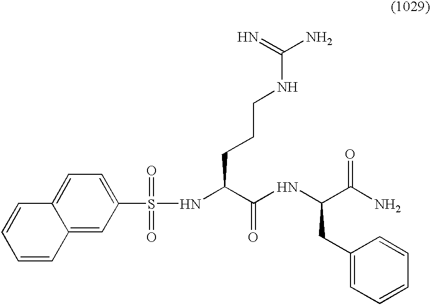

In another embodiment, the compound has the structure:

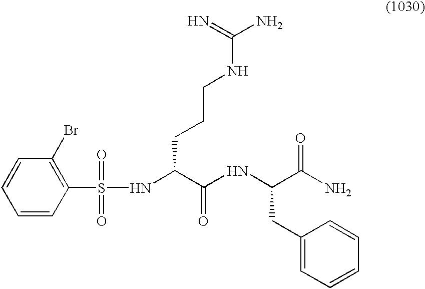

In another embodiment, the compound has the structure:

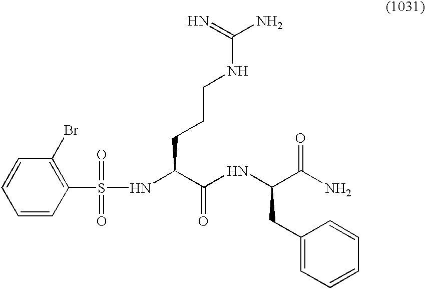

In another embodiment, the compound has the structure:

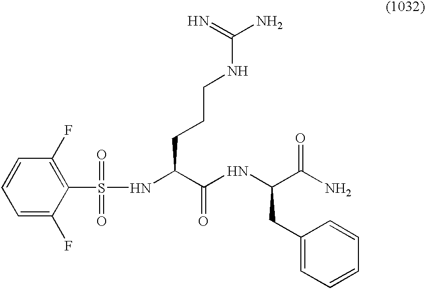

In another embodiment, the compound has the structure:

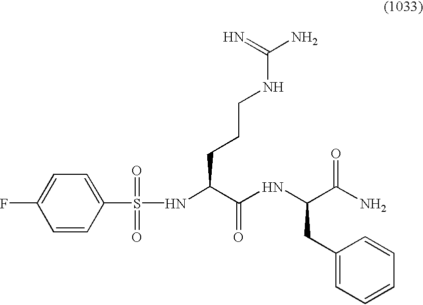

In another embodiment, the compound has the structure:

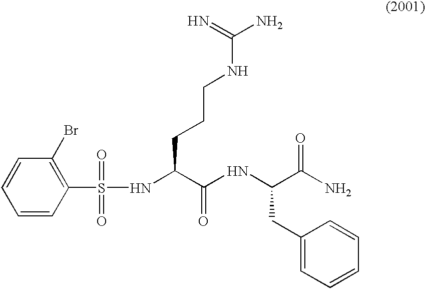

In another embodiment, the compound has the structure:

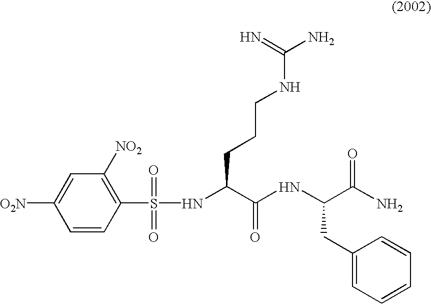

In another embodiment, the compound has the structure:

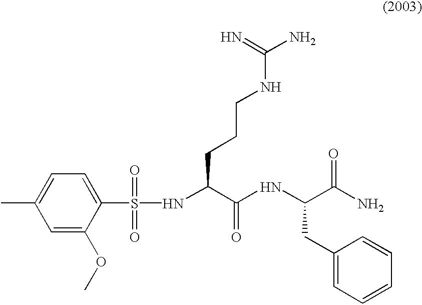

In another embodiment, the compound has the structure:

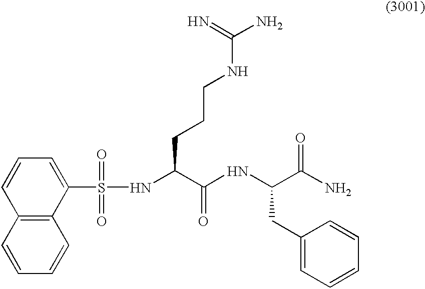

This invention also provides a pharmaceutical composition comprising any of the aforementioned compounds together with a pharmaceutically acceptable carrier.

This invention further provides a method of preparing a pharmaceutical composition comprising mixing any of the aforementioned compounds with a pharmaceutical acceptable carrier.

The carrier may be phosphate buffered saline, physiological saline or water, for example.

This invention further provides a compound which is converted in vivo to any of the aforementioned compounds.

This invention yet further provides a compound which is a metabolite of any of the aforementioned compounds.

This invention also provides a salt of any of the aforementioned compounds.

This invention further provides a method of treating pain in a subject in need of such treatment comprising administering to the subject an effective amount of any of the aforementioned compounds.

This invention yet further provides a method of treating a lower urinary tract disorder in a subject in need of such treatment comprising administering to the subject an effective amount of any of the aforementioned compounds. The lower urinary tract disorder may be interstitial cystitis, stress incontinence or urge incontinence.

For certain compounds, enantiomers, diastereomers, double bond stereoisomers and double bond regioisomers exist. Some compounds have multiple chiral centers, each of which can independently be either the R or the S configuration. This invention contemplates racemic mixtures of as well as isolated enantiomers, diastereomers, double bond stereoisomers and double bond regioisomers.

The invention also provides for each pure stereoisomer of any of the compounds described herein. Such stereoisomers may include enantiomers, disastereomers, or E or Z alkene isomers. The invention also provides for stereoisomeric mixtures, including racemic mixtures, diastereomeric mixtures, or E/Z isomeric mixtures. Stereoisomers can be synthesized in pure form (Nógrádi, M.; Stereoselective Synthesis, (1987) VCH Editor Ebel, H. and Asymmetric Synthesis, Volumes 3-5, (1983) Academic Press, Editor Morrison, J.) Or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J).

In addition the compounds of the present invention may be present as enatiomers, diasteriomers, isomers or two or more of the compounds may be present to form a racemic or diastereomeric mixture.

The compounds of the present invention are preferably 80% pure, more preferably 90% pure, and most preferably 95% pure.

As used herein, the term aryl is used to include phenyl, benzyl, or naphthyl, and the term hereroaryl is used to include pyrazinyl, imidazolyl, imidazolinyl, indolyl, benzimidazolyl, benzfuranyl, pyrimidinyl, benzothiophenyl, isoquinolyl, or quinolyl. The term arylalkyl is used to designate an C1-C6 alkyl chain substituted with an aryl group and the term heteroarylalkyl is used to designate a C1-C6 alkyl chain substituted with a heteroaryl group.

In the present invention, the term “heteroaryl” is used to include five and six membered unsaturated rings that may contain one or more oxygen, sulfur, or nitrogen atoms. Examples of heteroaryl groups include, but are not limited to, furanyl, thienyl, pyrroyl, oxazolyl, thiasolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, and triazinyl.

In addition the term “heteroaryl” is used to include fused bicyclic ring systems that may contain one or more heteroataoms such as oxygen, sulfur and nitrogen. Examples of such heteroaryl groups include, but are not limited to, indolizinyl, indolyl, isoindolyl, benzo[b]furanyl, benzo[b]thiophenyl, indazolyl, benzimidazolyl, purinyl, benaoxazolyl, benzisoxazolyl, benzo[b]thiazolyl, imidazo[2,1-b]thiazolyl, cinnolinyl, quinasolinyl, quinoxalinyl, 1,8-naphthyridinyl, pteridinyl, quinolinyl, isoquinolinyl, phthalimidyl and 2,1,3-benzothiazolyl.

Heterocyclic is defined as a 3 to 10 atom-ring containing at least one saturated bond and containing in any position one or more of the following atoms: N, O, S. Examples of heterocyclic rings include but are not limited to tetrahydrofuran, dihydrofuran, tetrahydropyran, kihydropyran piperidine, dihydropiperidine, pyrrolidine, dihydropyrrolidine dioxane, piperazin.

Data provided herein show that sulfonylamide compounds containing an arginine unit have agonist and/or antagonist activity at NPFF receptors. It is therefore reasonable to expect that replacement of arginine with lysine or known mimics of arginine will also provide agonists and/or antagonists of NPFF receptors. Such mimetic structures described herein are derived from commercially-available known mimics of arginine. One source is, RSP Amino Acid Analogs Inc., 1999 Building Block Index, Worcester, Mass. 01605, USA.

In separate embodiments, the abnormality is a lower urinary tract disorder such as interstitial cystitis or urge incontinence such as urge incontinence or stress incontinence, particularly urge incontinence, a regulation of a steroid hormone disorder, an epinephrine release disorder, a gastrointestinal disorder, irritable bowel syndrome, a cardiovascular disorder, an electrolyte balance disorder, diuresis, hypertension, hypotension, diabetes, hypoglycemia, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a serotonergic function disorder, an olfaction disorder, nasal congestion, a sympathetic innervation disorder, an affective disorder, pain, migrane, psychotic behavior, morphine tolerance, or addiction.

As used herein, the phrase “pharmaceutically acceptable carrier” means any of the standard pharmaceutically acceptable carriers. Examples include, but are not limited to, phosphate buffered saline, physiological saline, water, and emulsions, such as oil/water emulsions.

The formulations of the present invention can be solutions, suspensions, emulsions, syrups, elixirs, capsules, tablets, and the like. The compositions may contain a suitable carrier, diluent, or excipient, such as sterile water, physiological saline, glucose, or the like. Moreover, the formulations can also be lyophilized, and/or may contain auxiliary substances, such as wetting or emulsifying agents, pH buffering agents, adjuvants, gelling or viscosity enhancing additives, preservatives, flavoring agents, colors, and the like, depending upon the route of administration and the preparation desired. Standard texts, such as “Remington's Pharmaceutical Science”, 17th Ed., 1985, incorporated herein by reference, may be consulted to prepare suitable preparations, without undue experimentation.

The formulations can include powdered carriers, such as lactose, sucrose, mannitol, starch, cellulose derivatives, magnesium stearate, stearic acid, and the like. Further, tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. The formulations can also contain coloring and flavoring to enhance patient acceptance. The formulations can also include any of disintegrants, lubricants, plasticizers, colorants, and dosing vehicles.

In general, water, a suitable oil, saline, aqueous dextrose (glucose), and related sugar solutions and glycols such as propylene glycol or polyethylene glycols such as propylene glycol or polyethylene glycols are suitable carriers for parenteral solutions. Solutions for parenteral administration contain preferably a water soluble salt of the active ingredient, suitable stabilizing agents, and, if necessary, buffer substances.

Antioxidants such as, for example, sodium bisulfate, sodium sulfite, citric acid and its salts, sodium EDTA, ascorbic acid, and the like can be used either alone or in combination with other suitable antioxidants or stabilizing agents typically employed in the pharmaceutical compositions. In addition, parenteral solutions can contain preservatives, such as, for example, benzalkonium chloride, methyl- or propyl-paraben, chlorobutanol and the like.

The present invention includes within its scope prodrugs of the compounds of this inventions. In general, such prodrugs will be functional derivatives of the compounds of the invention which are readily convertible in vivo into the required compound.

Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs, ed. H. Bundgaard, Elsevier, 1985, the content of which is incorporated into the subject description by reference.

“Prodrugs” are considered to be any covalently bonded drug carriers that release the active parent drug upon in vivo administration. Prodrugs of molecules containing guanidines or other basic functional groups are prepared by modifying these groups in such a way that the modifications are cleaved in vivo to the parent compounds. Prodrugs also include compounds wherein hydroxyl, guanidino, amino, carboxy or sulfhydryl groups are ‘protected’ with any group that, upon administration to a mammalian subject, these functional groups are re-generated. Examples of prodrugs include, but are not limited to, acetate, formayl, benzoyl, polyethylene glycolyl derivatives of guanidinyl, amino, or alcohol compounds; phosphate esters, dimethylglycine esters, aminoalkylbenzyl esters, aminoalkyl esters and carbosyalkyl esters of alcholols and phenols, and various alkyl and aryl or polyethyleneglycol esters of carbosylic acids. In particular, a prodrug of a guanidino or amino group may contain an acyl group(s) attached to the basic nitrogen(s), forming an N-acyl derivative(s).

Included in this invention are pharmaceutically acceptable salts and complexes of all of the compounds described herein. The salts include, but are not limited to, the following acids and bases: Inorganic acids which include hydrochloric acid, hydrofluoric acid, hydrobromic acid, hydroiodic acid, sulfuric acid, and boric acid; organic acids which include acetic acid, trifluoroacetic acid, formic acid, oxalic acid, malonic acid, succinic acid, fumaric acid, tartaric acid, maleic acid, citric acid, methanesulfonic acid, trifluoromethanesulfonic acid, benzoic acid, glycolic acid, lactic acid, and mandelic acid; inorganic bases include ammonia and hydrazine; and organic bases which include methylamine, ethylamine, hydroxyethylamine, propylamine, dimethylamine, diethylamine, trimethylamine, triethylamine, ethylenediamine, hydroxyethylamine, morpholine, piperazine, and guanidine.

This invention further provides for the hydrates and polymorphs of all of the compounds described herein.

The present invention further includes metabolites of the compounds of the present invention. Metabolites include active species produced upon introduction of compounds of this invention into the biological milieu.

One skilled in the art will readily appreciate that appropriate biological assays will be used to determine the therapeutic potential of the claimed compounds for treating the above noted disorders.

This invention will be better understood from the Experimental Details which follow. However, one skilled in the art will readily appreciate that the specific methods and results discussed are merely illustrative of the invention as described more fully in the claims which follow thereafter.

Experimental Details

General Methods:

All solution-phase reactions were performed under an inert atmosphere (argon) and the reagents, neat or in appropriate solvents, were transferred to the reaction vessel via syringe and cannula techniques. The solid phase synthesis reactions were performed in vials using J-KEM heating shakers (Saint Louis, Mo.). All amino acid derivatives used as starting materials were purchased from Calbiochem-Novabiochem (San Diego, Calif.). Anhydrous solvents were purchased from Aldrich Chemical Company and used as received. The compounds described were named using ACD/Name program (version 2.51, Advanced Chemistry Development Inc., Toronto, Ontario, M5H2L3, Canada). The ¹H and ¹³C spectra were recorded at 300 and 75 MHz, respectively (QE-300 Plus by GE, Fremont, Calif.). Chemical shifts are reported in parts per million (ppm) and referenced with respect to the residual proton (i.e. CHCl₃, CHD₂OD) of the deuterated solvent. Splitting patterns are designated as s=singlet; d=doublet; t=triplet; q=quartet; p=quintet; sextet; septet; dd=doublet of a doublet; b=broad; m=multiplet. Elemental analyses were performed by Robertson Microlit Laboratories, Inc. Low-resolution electrospray mass spectra (ESMS) were measured on a Platform II instrument (Fisons, Manchester, UK) and MH⁺ is reported. Thin-layer chromatography (TLC) was carried out on glass plates precoated with silica gel 60 F₂₅₄ (0.25 mm, EM Separations Tech.). Preparative TLC was carried out on glass sheets precoated with silica gel GF (2 mm, Analtech). Flash column chromatography was performed on Merck silica gel 60 (230-400 mesh). The structures of the final products were confirmed by standard analytical methods such as elemental analysis and spectroscopic characteristics such as MS, NMR, analytical HPLC.

Synthesis:

The compounds of the present invention may be synthesized by the routes shown in Schemes 1 and 2, or with appropriate modifications as described herein. In Method 1, and Method 2, the product is isolated at the end of the synthesis, and purified by a suitable procedure such as high performance liquid chromatography (HPLC), crystallization, column chromatography, thin layer chromatography, etc. While preferred reactants have been identified herein, it is further contemplated that the present invention would include chemical equivalents to each reactant(s) specifically enumerated in this disclosure.

Two general procedures were used in the synthesis of the specific sulfonamides described above. They are described by using 1-naphthalenesulfonylamido-Arg-Phe-amide as an example:

Method I: Solid Phase Synthesis:

The general scheme for the solid phase synthesis is shown in Scheme 1.

General Experimental Procedure:

Rink amide MBHA resin (1.85 g, 1 mmol, 0.54 mmol/g, Novabiochem, San Diego, Calif., #01-64-0013) was swelled in a mixture of N,N-dimethylformamide (DMF), and N-methylpyrrolidone (NMP) (1:1, 25 mL) in a glass column with a sintered glass frit, on a platform shaker, for 10 min. The solvents were drained and the resin was treated with 30% piperidine in DMF (25 mL) for 5 min. and the liquid was drained. The piperidine treatment was repeated for 25 min. The resin was then washed, for 5 min. per wash, with DMF:NMP (1:1, 25 mL, three times), followed by methanol (25 mL, two times) and DMF:NMP (1:1, 25 mL, three times). The resin was then treated with a pre-mixed solution of Fmoc-L-phenylalanine (1.549, 4 mmol), HBTU (1.59, 4 mmol) and diisopropylethylamine (1.4 mL, 8 mmol). The resin slurry was shaken for 2 h. After draining of the amino acid solution, the resin was washed three times with DMF:NMP (1:1, 25 mL). The resin was treated with 30% piperidine in DMF (25 mL) for 5 min. and the liquid was drained. The piperidine treatment was repeated for 25 min. The resin was then washed, for 5 min. per wash with DMF:NMP (1:1, 25 mL, three times), followed by methanol (25 mL, two times) and DMF:NMP (1:1, 25 mL, three times). The resin was then treated with a pre-mixed solution of Fmoc-L-arginine(Pbf) (2.69, 4 mmol) with HBTU (1.5 g, 4 mmol) and diisopropylethyl amine (1.4 mL, 8 mmol). The resin slurry was shaken for 2 h. After draining of the amino acid solution, the resin was washed three times with DMF:NMP (1:1, 25 mL). The resin was treated with 30% piperidine in DMF (25 mL) for 5 and 25 min, respectively, as described above. The resin was then washed, for 5 min. each, with DMF:NMP (1:1, 25 mL, three times), followed by methanol (25 mL, two times) and DMF:NMP (1:1, 25 mL, three times). To the resin was then added 1-naphthalenesulfonyl chloride (0.53 g, 2 mmol), and triethylamine (0.56 mL, 4 mmol) in DMF (10 mL). After shaking for 3 h, the reagents were drained, and the resin was washed for 5 min. per wash, with DMF:NMP (1:1, 25 mL, three times), followed by methanol (25 mL, two times) and vacuum dried. The product was cleaved from the resin with trifluoroacetic acid:dithioethane:anisole thioanisole:m-cresol:water:triisopropylsilane (78:5:3:3:3:5:3, 25 mL) for 2 h and the cleavage solution was filtered. The filtrate was evaporated to an oil, and anhydrous ether was added to precipitate the product, which was filtered, washed with ether, and vacuum dried to yield the crude product (286 mg, 45.6%). The product was purified by using reverse phase preparative HPLC (250×22.5 mm, Primesphere C18-HC) with a gradient of 10%-70% acetonitrile (0.1% TFA) in water (0.1% TFA) over 30 min (25 mL/min flow rate, detection at 215 nm). The fractions containing the product were pooled and lyophilized to yield the product (107 mg).

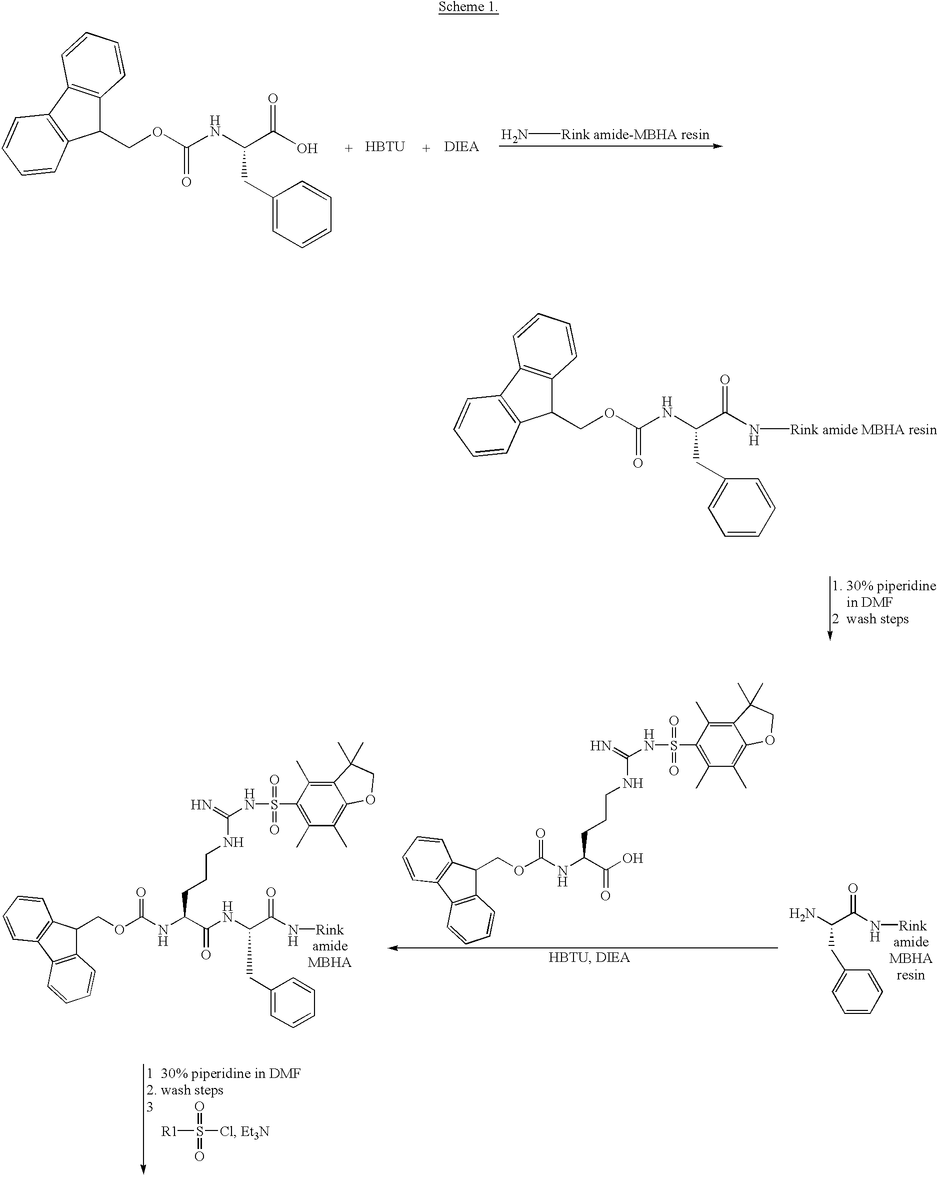

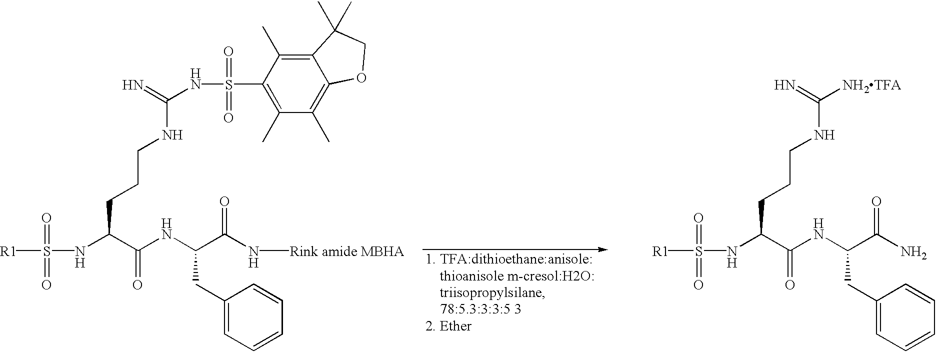

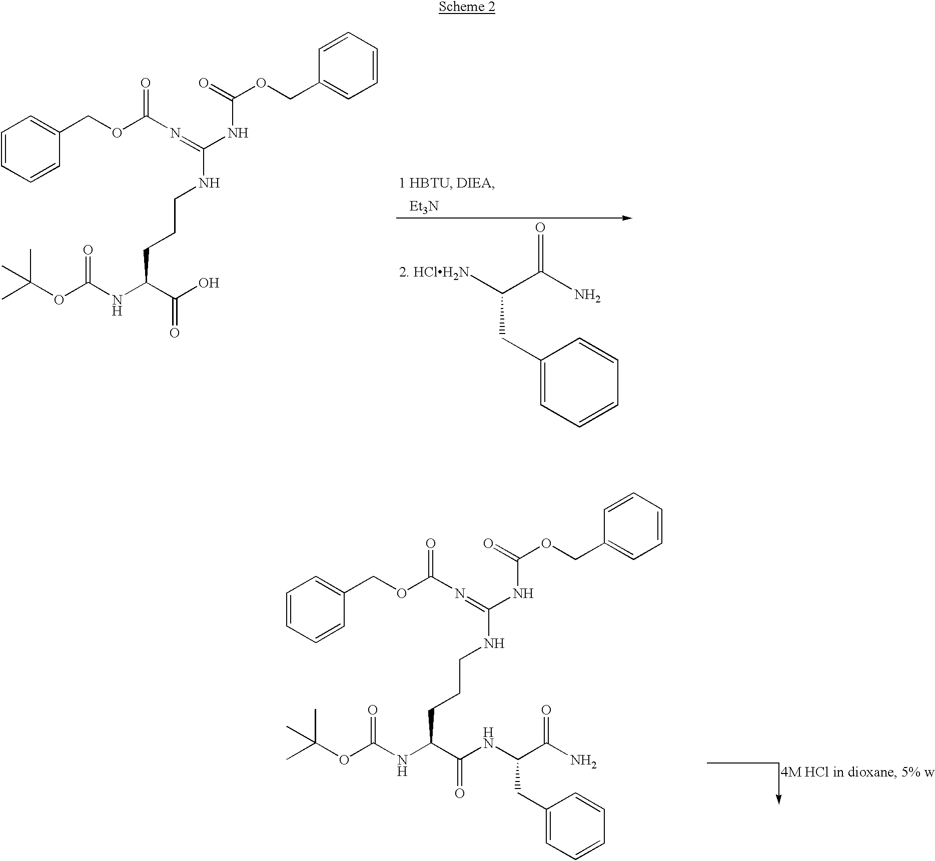

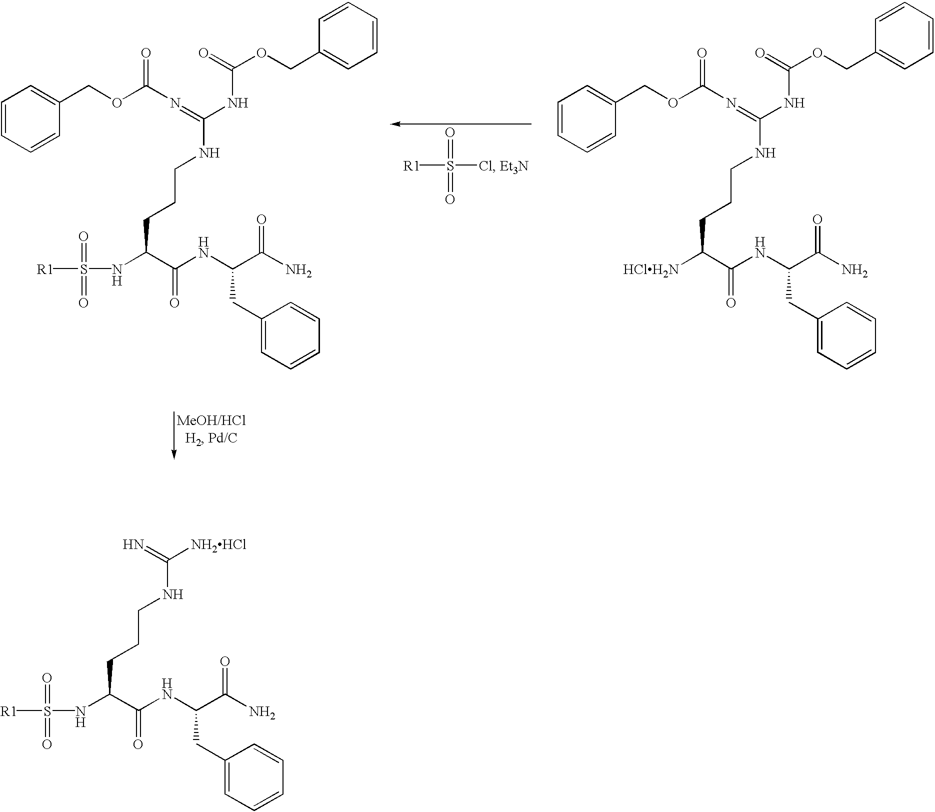

Method 2. Solution-Phase Synthesis.

Experimental Procedures for Method 2.

(N^{α}-Boc)arginine(diZ)-phenylalaninamide: (Z=benzyloxy carbonyl):

(N^{α}-Boc)-arginine(diZ)-OH (4.89, 8.85 mmol) was suspended in dichloromethane (100 mL), and N,N-dimethylformamide (DMF) was added dropwise while stirring, until a clear solution was obtained (10 mL). To this solution was added HBTU (3.4 g, 8.85 mmol) in DMF (20 mL). Triethylamine (1.3 mL, 8.85 mmol) was added and the solution was stirred for 5 min. To this was added a mixture of L-phenylalaninamide.HCl (1.8 g, 8.85 mmol) in dichloromethane (25 mL), containing triethylamine (3.7 mL, 26.55 mmol). The reaction mixture was stirred overnight. The volatiles were evaporated in a rotary evaporator at 45° C. The residue was dissolved in ethylacetate (200 mL) and washed with water, saturated aq. NaHCO₃, water, sat. aq. NaCl and dried (Na₂SO₄). Evaporation of the solvent gave the crude product, which was crystallized from ethyl acetate: 5.4 g (90%); m.p. 122-124° C. (dec.);

H-Arginine(diZ)-phenylalaninamide.HCl:

(N^{α}-Boc)arginine(diZ)-phenylalaninamide (3.3 g), was dissolved in THF (20 mL), and treated with 4M HCl in dioxane (20 mL) for 20 min. The solvent was evaporated to dryness. The residue was treated with anhydrous ether and triturated. The precipitated product was filtered and washed with ether, and vacuum dried: 2.15 g (72%).

In the final step, 1-naphthalenesulfonyl chloride (2 eq.) was coupled with H-Arginine(diZ)-phenylalaninamide.HCl, with 4 eq. of triethylamine in THF for 4-6 h. The reaction mixtur was evaporated to dryness, and partitioned between ethyl acetate and sat. aq. NaHCO₃. The ethyl acetate layer was washed with water, sat. aq. NaCl and dried (Na₂SO₄). Filtration and evaporation of the ethyl acetate led to the protected compound. The Z groups were removed by hydrogenation with Pd/C (5%) as the catalyst, in ethanol, with 0.5% V/V conc. HCl. The product was purified by using reverse phase preparative HPLC (250×22.5 mm, Primesphere C18-HC) with a gradient of 10%-70% acetonitrile (0.1% TFA) in water (0.1% TFA) over 30 min (25 mL/min flow rate, detection at 215 nm). The fractions containing the product were pooled and lyophilized to yield the product.

The synthesis of N-amido-substituted products (where R3 and R4 in the generic structure is a substituent other than H), can be achieved by modifying procedure 1 to accommodate the incorporation of R3 or R4 via alkylation or reductive coupling. After the coupling of the first residue (e.g., Fmoc Phenylalanine in the general procedure) to the resin followed by the removal of the Fmoc protecting group as described above, the resin is treated with the appropriate alkyl halide (0.9 eq.), in DMF or dichloromethane, with 2-3 eq. of triethylamine for 3-4 h. Alternately, reductive coupling with the appropriate aldehyde as described in the literature (Gordon, D. W. and Steele, J., Bioorg. Med. Chem. Lett., 5(1), 1995, 47-50), can be utilized to incorporate R4. In the next step, Fmoc-Arginine(Pbf) is coupled to the secondary amine on resin, and the Fmoc protecting group removed, again as described in the general procedure. Then, the R3 group can be introduced by methods described above, followed by the coupling of the appropriate sulfonyl chloride. Cleavage with the trifluoroacetic acid cocktail and precipitation with ether gives the purified product, which can be purified by preparative HPLC as described above.

In schemes 1 and 2, the protected forms of phenylalanine and arginine can each be replaced with appropriately protected forms of other amino acids (which can be obtained from RSP Amino Acid Analogs Inc., Boston, Mass.), in order to obtain the claimed compounds. Compounds where R2 is —(CH₂)ₙN(R7)₂ wherein at least one R7 group H can be synthesized by using the appropriate amino acids as described above, followed by protecting group cleavage and treatment of the product with the appropriate alkylating agent(s) R7-X, (where X═Cl, Br, I), with an excess of a tertiary amine base, in a polar solvent.

For compounds where R5═OH, the synthesis can be achieved by starting with the protected phenylalanine attached to Wang resin or 2-chlorotrityl chloride resin. Cleavage with the TFA cocktail after the synthesis is complete gives the product with the C-terminal acid. For the synthesis of compounds with R5═N(R8)₂, it is preferred to first obtain the fully-protected sulfonylated compound as follows: The synthesis is performed by starting with Fmoc-phenylalanine attached to 2-chlorotritylchloride resin. Upon completion of the synthesis, the protected compound is obtaining by cleaving it from the resin with 1% TFA in dichloromethane. The cleavage solution is neutralized with pyridine in methanol, and evaporated. The crude compound containing a C-terminal acid is then coupled to an appropriate amine ((R8)₂NH) by using a coupling procedure similar to that described in Method 2, to give the substituted amide.

Compound 1001

N1-[(1S)-2-Amino-1-benzyl-2-oxoethyl]-(2S)-(5-guanidino)-2-[(1-naphthylsulfonyl)amino]pentanamide (1). (Alternate name: 1-naphthalenesulfonylamido-Arg-Phe-NH₂).

This compound was synthesized according to Method 1 described above.

Data: ESMS 511(MH+); ¹H NMR (CD₃OD) δ 8.65 (d, J=8.1 Hz, 1H), 8.13 (t, J=6.9 Hz, 2H), 8.01 (m, 2H), 7.64 (m, 2H), 7.52 (t, J=9.0 Hz), 7.05-7.2 (m, 4H), 4.30 (q, J=6.3, 6.0 Hz, 1H), 3.59 (m, 1H), 2.91 (dd, J=7.2, 9.6 Hz), 2.79 (m, 2H), 2.63 (m, 1H), 1.43 (m, 2H), 1.25 (m, 1H), 1.16 (m, 1H); ¹³C NMR (CD₃OD) d 24.86, 30.07, 37.85, 40.67, 54.69, 56.66, 104.75, 124.49, 124.51, 126.98, 127.28, 128.43, 128.59, 129.34, 134.98, 137.36, 158.02, 172.28, 174.77;

Anal. C₂₅H₃₀N₆O₄S+1.75 CF₃COOH calcd. C, 48.20%; H, 4.51%; N, 11.83%; S, 4.52%; found C, 48.08%; H, 4.51%; N, 11.91%; S, 4.64%; [a]_{D}=−29.8 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 18.9 min;

Compound 1002

N1-[(1S)-2-Amino-1-benzyl-2-oxoethyl]-(2S) -{[amino(imino)methyl]amino}-2-[(3-nitrophenyl)sulfonyl]amino}pentanamide (2).

(Alternate name: 3-Nitrophenylsulfonylamido-Arg-Phe-NH₂).

This compound was synthesized as described in Method 1, except that 3-nitrophenylsulfonyl chloride (442 mg, 2 mmol) was used in place of 1-naphthalenesulfonyl chloride.

Data: ESMS 506(MH+);

Compound 1003

N1-[(1S)-2-Amino-1-benzyl-2-oxoethyl]-(2S) -{[amino(imino)methyl]amino}-2-[(4-nitrophenyl)sulfonyl]amino}pentanamide (3).

(Alternate name: 4-Nitrophenylsulfonylamido-Arg-Phe-NH₂).

This compound was synthesized as described in Method 1, except that 4-nitrophenylsulfonyl chloride (442 mg, 2 mmol) was used in place of 1-naphthalenesulfonyl chloride.

Data: ESMS 506(MH+);

Compound 1004

N1-[(1S)-2-Amino-1-benzyl-2-oxoethyl]-(2S) -{[amino(imino)methyl]amino}-2-[(2,6-difluorophenyl)sulfonyl]amino}pentanamide (4).

(Alternate name: 2,6-Difluorophenylsulfonylamido-Arg-Phe-NH₂).

This compound was synthesized as described in Method 1, except that 2,6-dichlorophenylsulfonyl chloride (425.2 mg, 2 mmol) was used in place of 1-naphthalenesulfonyl chloride.

Data: ESMS 497(MH+);

Compound 1005

N1-[(1S)-2-Amino-1-benzyl-2-oxoethyl]-(2S) -{[amino(imino)methyl]amino}-2-[(4 -fluorophenyl)sulfonyl]amino}pentanamide (5).

(Alternate name: 4-Fluorophenylsulfonylamido-Arg-Phe-NH₂).

This compound was synthesized as described in Method 1, except that 4-fluorophenylsulfonyl chloride (389.2 mg, 2 mmol) was used in place of 1-naphthalenesulfonyl chloride.

Data: ESMS 479(MH+);

Compound 1006

N1-[(1S)-2-Amino-1-benzyl-2-oxoethyl]-(2S) -{[amino(imino)methyl]amino}-2-[(4-chlorophenyl)sulfonyl]amino}pentanamide (6).

(Alternate name: 4-Chlorophenylsulfonylamido-Arg-Phe-NH₂).

This compound was synthesized as described in Method 1, except that 4-chlorophenylsulfonyl chloride (422.14 mg, 2 mmol) was used in place of 1-naphthalenesulfonyl chloride.

Data: ESMS 495(MH+);

Compound 2001

N1-[(1S)-2-Amino-1-benzyl-2-oxoethyl]-(2S) -{[amino(imino)methyl]amino}-2-[(2-bromophenyl)sulfonyl]amino}pentanamide (7).

(Alternate name: 2-Bromophenylsulfonylamido-Arg-Phe-NH₂).

This compound was synthesized as described in Method 1, except that 2-bromophenylsulfonyl chloride (511.04 mg, 2 mmol) was used in place of 1-naphthalenesulfonyl chloride.

Data: ESMS 539(MH+);

Compound 1007

N1-[(1S)-2-Amino-1-benzyl-2-oxoethyl]-(2S) -{[amino(imino)methyl]amino}-2-[(p-tolyl)sulfonyl]amino}pentanamide (8).

(Alternate name: p-Tolylsulfonylamido-Arg-Phe-NH₂).

This compound was synthesized as described in Method 1, except that 4-methylphenylsulfonyl chloride (381.3 mg, 2 mmol) was used in place of 1-naphthalenesulfonyl chloride.

Data: ESMS 475(MH+);

Compound 1008

N1-[(1S)-2-Amino-1-benzyl-2-oxoethyl]-(2S) -{[amino(imino)methyl]amino}-2-[phenylsulfonyl]amino}pentanamide (9).

(Alternate name: Phenylsulfonylamido-Arg-Phe-NH₂)—

This compound was synthesized as described in Method 1, except that phenylsulfonyl chloride (353.24 mg, 2 mmol) was used in place of 1-naphthalenesulfonyl chloride.

Data: ESMS 461(MH+);

Compound 1009

N1-[(1S)-2-Amino-1-benzyl-2-oxoethyl]-(2S) -{[amino(imino)methyl]amino}-2-[(4-methoxyphenyl)sulfonyl]amino}pentanamide (10).

(Alternate name: 4-Methoxyphenylsulfonylamido-Arg-Phe-NH₂).

This compound was synthesized as described in Method 1, except that 4-methoxyphenylsulfonyl chloride (413.3 mg, 2 mmol) was used in place of 1-naphthalenesulfonyl chloride.

Data: ESMS 491 (MH+)

Compound 1010

N1-[(1S)-2-Amino-1-benzyl-2-oxoethyl]-(2S) -{[amino(imino)methyl]amino}-2-[(2,4-dichlorophenyl)sulfonyl]amino}pentanamide (11).

(Alternate name: 2,4-Dichlorophenylsulfonylamido-Arg-Phe-NH₂).

This compound was synthesized as described in Method 1, except that 2,4-dichlorophenylsulfonyl chloride (491.02 mg, 2 mmol) was used in place of 1-naphthalenesulfonyl chloride.

Data: ESMS 529(MH+); ¹H NMR (CD₃OD) d 8.13 (d, J=7.88 Hz, 1H), 7.87 (d, J=8.4 Hz, 1H), 7.61 (d, J=2.02 Hz, 1H), 7.37 (dd, J=2.7, 3.7 Hz, 2H), 7.25 (m,4H), 4.35 (m, 1H), 3.75 (q, J=1.77, 5.75 Hz, 1H), 3.04(m, 2H), 2.96(m, 1H), 2.78 (m, 1H) 1.44-1.65 (m, 4H); ¹³C NMR (CD₃OD) d 25.01, 30.42, 38.09, 40.93, 54.90, 56.78, 127.05, 127.77, 128.69, 129.49, 131.84, 132.41, 133.46, 139.71, 157.79, 171.84, 174.84; [a]_{D}=+7.0 (c =1% W/V in methanol);

Anal. C₂₁H₂₆Cl₂N₆O₄S+1.5 CF₃COOH calc. C, 41.15%; H, 3.96%; N, 12.00%; Cl, 10.12%; S, 4.58%; found C, 41.46%; H, 4.00%; N, 12.37%; Cl, 9.98%; S, 4.80%;

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.9 min;

Compound 1011

N1-[(1S)-2-Amino-1-benzyl-2-oxoethyl]-(25)-5-[amino(imino)methyl]amino-2-[(benzylsulfonyl)amino]pentanamide (12).

Alternate name: a-Toluenesulfonamido-Arg-Phe-NH2

This compound was synthesized as described in Method 1, except that a-toluenesulfonyl chloride (379.3 mg, 2 mmol) was used in place of 1-naphthalenesulfonyl chloride.

Data: ESMS 475(MH+); ¹H NMR (CD₃OD) d 7.317-7.16 (m, 10H), 7.06 (t, J=8.0 Hz, 1H), 4.69 (q, J=5.0, 4.8 Hz, 1H), 4.11 (m, 2H), 3.75 (m, 2H), 3.17(m, 1H), 3.05 (t, J=6.9 Hz, 2H) 2.87 (m, 2H), 1.55 (m, 2H), 1.44 (m, 2H), 1.28 (t, J=7.3 Hz,1H); ¹³C NMR (CD₃OD) d 8.38, 24.96, 30.60, 38.04, 40.95, 54.75, 56.92, 58.92, 104.98, 127.06, 128.71, 128.73, 129.48, 129.87, 131.28, 137.74, 157.83, 172.83, 175.21;[a]_{D}=−5.0 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 21.7 min;

Compound 1012

N1-[(1S)-2-Amino-1-benzyl-2-oxoethyl]-(2S)-{[amino(imino)methyl]amino}-2-[4-iodophenyl)sulfonyl]amino}pentanamide (13).

(Alternate name: 4-Iodophenylsulfonylamido-Arg-Phe-NH₂).

This compound was synthesized as described in Method 1, except that 4-iodophenylsulfonyl chloride (605.04 mg, 2 mmol) was used in place of 1-naphthalenesulfonyl chloride.

Data: ESMS 506(MH+);586.99¹H NMR (CD₃OD) d 1.29 (t, J=7.3 Hz, 1H), 1.44 (m, 2H), 1.55 (m, 2H), 2.73 (dd, J=8.8, 4.9 Hz, 1H), 3.02 (m, 2H), 3.20 (q, 1H), 3.71 (t, J=6 Hz, 1H), 4.3 (q, J=6.0, 2.86 Hz), 7.34 (m, 5H), 7.45 (d, J=8.6 Hz, 2H), 7.80 (d, J=8.6 Hz, 2H); [a]_{D}=+5.7 (c=1% W/V in methanol);

Anal. C₂₁H₂₇IN₆O₄S+1.25 CF₃COOH calcd. C, 38.72%; H, 3.91%; N, 11.53%; S, 4.40%; found C, 38.51%; H, 3.75%; N, 11.07%; S, 4.49%;

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.7 min;

Compound 1013

N1-[(1S)-2-Amino-1-benzyl-2-oxoethyl]-(2S)-{[amino(imino)methyl]amino}-2-[(2-thiophene)sulfonyl]amino}pentanamide (14).

(Alternate name: 2-Thiophenesulfonylamido-Arg-Phe-NH₂).

This compound was synthesized as described in Method 1, except that 2-thiophenesulfonyl chloride (365.3 mg, 2 mmol) was used in place of 1-naphthalenesulfonyl chloride.

Data: ESMS 467(MH+); ¹H NMR (CD₃OD) d 1.282 (t, J=7.3 Hz, 1H), 1.35 (m, 2H), 1.37 (m, 2H), 2.91 (m, 1H), 2.99 (t, J=7.0 Hz, 2H), 3.08-3.31 (m, 2H), 3.73 (t, J=5.9 Hz 1H), 4.44 (t, J=5.5 Hz, 1H), 7.01 (t, 3.8 Hz, 1H), 7.20-2.28 (m, 6H), 7.47 (q, J=2.5, 1.2 Hz, 1H), 7.69 (q, J=3.7, 1.2 Hz, 1H); [a]_{D}=−5.9 (c=1% W/V in methanol);

HPLC Primesphere C-18 reverse phase column, 4.6×250 mm, 10-56% acetonitrile (0.1% TFA) in water (0.1% TFA) over 24 14.9 min;

Compound 1014

N1-[(1S)-2-Amino-1-benzyl-2-oxoethyl]-(2S)-(5-guanidino)-2-[(2-naphthylsulfonyl)amino]pentanamide (15). (Alternate name: 2-naphthalenesulfonylamido-Arg-Phe-NH₂).

This compound was synthesized as described in Method 1, except that 2-naphthalenesulfonyl chloride (453.36 mg, 2 mmol) was used in place of 1-naphthalenesulfonyl chloride.

Data: ESMS 511(MH+); ¹H NMR (CD₃OD) d 1.28 (t, J=7.3 Hz, 1H), 1.37 (m, 2H), 1.52 (m, 2H), 2.48 (q, J=8.3, 8.4 Hz, 1H), 2.86 (t, J=6.6 Hz, 1H), 2.93 (m, 2H), 3.10 (q, J=7 Hz, 1H), 3.69 (q, J=6.2, 1.4 Hz, 1H), 4.25 (q, J=6.7, 1.5 Hz, 1H), 7.01 (m, 2H), 7.16 (m, 3H), 7.63 (m, 2H), 7.7 (d, J=6.8, 1.8 Hz, 1H), 7.98 (m, 3H), 8.39 (s, 1H); ¹³C NMR (CD₃OD) d 25.00, 30.63, 38.01, 40.93, 54.90, 56.69, 56.72, 122.29, 127.08, 127.22, 127.34, 128.67, 129.46, 130.99, 131.06, 131.05, 132.78, 132.85, 132.91, 137.96, 142.92, 148.77, 157.79, 171.71, 174.82;

Anal. C₂₅H₃₀N₆O₄S+1.25 CF₃COOH calcd. C, 50.57%; H, 4.82%; N, 12.87%; S, 4.91%; found C, 50.74%; H, 4.98%; N, 12.79%; S, 4.76%; [a]_{D}=−9.2 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.0 min;

Compound 1015

N1-[(1S)-2-Amino-1-benzyl-2-oxoethyl]-(2S)-{[amino(imino)methyl]amino}-2-[3, dimethoxyphenyl)sulfonyl]amino}pentanamide (16).

(Alternate name: 3,4-Dimethoxyphenylsulfonylamido-Arg-Phe-NH₂).

This compound was synthesized as described in Method 1, except that 3,4-dimethoxyphenylsulfonyl chloride (473.36 mg, 2 mmol) was used in place of 1-naphthalenesulfonyl chloride.

Data: ESMS 521(MH+); ¹H NMR (CD₃OD) d 1.26 (m, 2H), 1.46 (m, 2H), 2.72 (dd, J=8.5, 5.3 Hz, 1H), 3.00 (t, J=8 Hz, 2H), 3.06 (m, 2H), 3.59 (q, J=1.3, 6.1 Hz, 1H), 3.83 (s, 3H), 3.85 (s, 3H), 4.4 (q, J=2.3, 6.2 Hz, 1H), 6.96 (d, J=8.4 Hz, 1H), 7.15-7.3 (m, 5H), 7.3 (m, 1H), 7.37 (dd, J=6.4, 2.0 Hz, 1H); Anal. C₂₃H₃₂N₆O₆S+1.2 CF₃COOH calcd. C, 46.40%; H, 5.09%; N, 12.78%; S, 5.05%; found C, 46.62%; H, 4.98%; N, 12.91%; S, 4.86%; [a]_{D}=−5.3 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 14.9 min;

Compound 1016

N1-[(1S)-2-Amino-1-benzyl-2-oxoethyl]-(2S)-{[amino(imino)methyl]amino}-2-[4-chloro-3-nitrophenyl)sulfonyl]amino}pentanamide (17).

(Alternate name: 4-Chloro-3-nitrophenylsulfonylamido-Arg-Phe-NH₂).

This compound was synthesized as described in Method 1, except that 4-chloro-3-nitrophenylsulfonyl chloride (512.14 mg, 2 mmol) was used in place of 1-naphthalenesulfonyl chloride.

Data: ESMS 540(MH+); ¹H NMR (CD₃OD) d 1.29 (t, J=7.3 Hz, 1H), 1.46-1.65 (m, 4H), 2.73 (dd, J=4.8, 8.6 Hz, 1H), 3.01 (dd, J=7, 8.7, 1H), 3.18 (m, 2H), 3.2 (q, J=6.2, 0.8 Hz, 1H), 4.3 (q, J=2.2, 6.3 Hz, 1H), 7.25 (m, 5H), 7.59 (d, J=8.6 Hz, 1H), 7.81 (dd, J=6.4, 1.2 Hz, 1H), 8.3 (m, 1H); Anal. C₂₁H₂₆ClN₇O₆S+1.5 CF C₃OOH calcd. C, 40.54%; H, 3.90%; Cl, 4.99%; N, 13.79%; S, 4.51%; found C, 40.45%; H, 3.73%; Cl, 4.99%; N, 13.76%; S, 4.96%; [a]_{D}=+34.1 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.9 min;

Compound 2002

N1-[(1S)-2-Amino-1-benzyl-2-oxoethyl]-(2S)-{[amino(imino)methyl]amino}-2-[2,4-dinitrophenyl)sulfonyl]amino pentanamide.

(Alternate name: 2,4-Dinitrophenylsulfonylamido-Arg-Phe-NH₂) (18).

This compound was synthesized as described in Method 1, except that 2,4-dinitrophenylsulfonyl chloride (533.24 mg, 2 mmol) was used in place of 1-naphthalenesulfonyl chloride.

Data: ESMS 550.9(MH+); ¹H NMR (CD₃OD) d 1.29 (t, J=7.3 Hz, 1H), 1.41 (m, 2H), 1.59 (m, 2H), 2.75 (dd, J=4.4, 9.5 Hz, 1H), 3.00 (dd, J=5.3, 5.2 Hz, 1H), 3.18 (m, 2H), 4.03 (q, J=2.3, 2.9 Hz, 1H), 4.25 (q, J=2.9, 3.0 Hz, 1H), 7.2 (m, 5H), 8.02 (d, J=4.0 Hz, 1H), 8.29 (dd, J=6.4, 2.2 Hz, 1H), 8.62 (d, J=2.2 Hz, 1H); Anal. C₂₁H₂₆N₈O₈S+1.275 CF₃COOH calcd. C, 40.65%; H, 3.95%; N, 16.10%; S, 4.61%; found C, 40.81%; H, 3.78%; N, 15.86%; S, 3.84%; [a]_{D}=−25.7 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.9 min;

Compound 1017

N1-[(1S)-2-Amino-1-benzyl-2-oxoethyl]-(2S)-{[amino(imino)methyl]amino}-2-[(3-chloro-4-fluorophenyl)sulfonyl]amino}pentanamide (19).

(Alternate name: 3-Chloro-4-fluorophenylsulfonylamido-Arg-Phe-NH₂).

This compound was synthesized as described in Method 1, except that 3-chloro-4-fluorophenylsulfonyl chloride (458.12 mg, 2 mmol) was used in place of 1-naphthalenesulfonyl chloride.

Data: ESMS 513 (MH+);

Compound 1018

N1-[(1S)-2-Amino-1-benzyl-2-oxoethyl]-(2S)-{[amino(imino)methyl]amino}-2-[(2-nitro-(4-trifluoromethyl)phenyl)sulfonyl]amino}pentanamide (20).

(Alternate name: 2-Nitro-4-trifluoromethyl phenylsulfonylamido-Arg-Phe-NH₂).

This compound was synthesized as described in Method 1, except that 2-Nitro-4-trifluoromethylphenylsulfonyl chloride (579.24 mg, 2 mmol) was used in place of 1-naphthalenesulfonyl chloride.

Data: ESMS 574 (MH+); [a]_{D}=−32.9 (c=1% W/V in methanol);

Compound 1019

N1-[(1S)-2-Amino-1-benzyl-2-oxoethyl]-(2S)-{[amino(imino)methyl]amino}-2-[(2,6-dichlorophenyl)sulfonyl]amino}pentanamide (21).

(Alternate name: 2,6-Dichlorophenylsulfonylamido-Arg-Phe-NH₂).

This compound was synthesized as described in Method 1, except that 2,6-dichlorophenylsulfonyl chloride (491.02 mg, 2 mmol) was used in place of 1-naphthalenesulfonyl chloride.

Data: ESMS 529 (MH+); [a]_{D}=−5.9 (c=1% W/V in methanol);

Compound 1020

N1-[(1S)-2-Amino-1-benzyl-2-oxoethyl]-(2S)-{[amino(imino)methyl]amino}-2-[3-(2,5-dichlorothiophene)sulfonyl]amino}pentanamide (22).

(Alternate name: 3-(2,5-Dichlorothiophene)sulfonylamido-Arg-Phe-NH₂).

This compound was synthesized as described in Method 1, except that 3-(2,5-dichlorothiophene)sulfonyl chloride (503.08 mg, 2 mmol) was used in place of 1-naphthalenesulfonyl chloride.

Data: ESMS 535, 536(MH+); [a]_{D}=+1.9 (c=1% W/V in methanol);

Compound 2003

N1-[(1S)-2-Amino-1-benzyl-2-oxoethyl]-(2S)-{[amino(imino)methyl]amino}-2-[(3-methyl-6-methoxyphenyl)sulfonyl]amino}pentanamide (23).

(Alternate name: 3-Methyl-6-methoxyphenylsulfonylamido-Arg-Phe-NH₂).

This compound was synthesized as described in Method 1, except that 3-methyl-6-methoxyphenylsulfonyl chloride (441.36 mg, 2 mmol) was used in place of 1-naphthalenesulfonyl chloride.

Data: ESMS 505(MH+); [a]_{D}=−1.6 (c=1% W/V in methanol);

Compound 1021

N1-[(1S)-2-Amino-1-benzyl-2-oxoethyl]-(25)-{[amino(imino)methyl]amino}-2-[(2,5-dichlorophenyl)sulfonyl]amino}pentanamide (24).

(Alternate name: 2,5-Dichlorophenylsulfonylamido-Arg-Phe-NH₂).

This compound was synthesized as described in Method 1, except that 2,5-dichlorophenylsulfonyl chloride (491.02 mg, 2 mmol) was used in place of 1-naphthalenesulfonyl chloride.

Data: ESMS 529, 530(MH+); [a]_{D}=−0.3 (c=1% W/V in methanol);

Compound 1022

N1-[(1S)-2-Amino-1-benzyl-2-oxoethyl]-(2S)-{[amino(imino)methyl]amino}-2-[3,4-dichlorophenyl)sulfonyl]amino}pentanamide (25).

This compound was synthesized as described in Method 1, except that 3,4-dichlorophenylsulfonyl chloride (491.02 mg, 2 mmol) was used in place of 1-naphthalenesulfonyl chloride.

Data: ESMS 528(MH+); [a]_{D}=+12.9 (c=1% W/V in methanol);

Compound 1023

N1-[(1S)-2-Amino-1-benzyl-2-oxoethyl]-(2S)-{[amino(imino)methyl]amino}-2-[3-cyanophenyl)sulfonyl]amino}pentanamide (26).

(Alternate name: 3-Cyanophenylsulfonylamido-Arg-Phe-NH₂).

This compound was synthesized as described in Method 1, except that 3-cyanophenylsulfonyl chloride (403.26 mg, 2 mmol) was used in place of 1-naphthalenesulfonyl chloride.

Data: ESMS 486(MH+); [a]_{D}=+14.9 (c=1% W/V in methanol);

Compound 1024

N1-[(1S)-2-Amino-1-benzyl-2-oxoethyl]-(2S)-{[amino(imino)methyl]amino}-2-[pentafluorophenyl)sulfonyl]amino}pentanamide (27).

(Alternate name: Pentafluorophenylsulfonylamido-Arg-Phe-NH₂).

This compound was synthesized as described in Method 1, except that pentafluorophenylsulfonyl chloride (533.14 mg, 2 mmol) was used in place of 1-naphthalenesulfonyl chloride.

Data: ESMS 550(MH+); [a]_{D}=+25.1(c=1% W/V in methanol);

Compound 1025

N1-[(1S)-2-Amino-1-benzyl-2-oxoethyl]-(25)-{[amino(imino)methyl]amino}-2-[5-bromo-2-methoxyphenyl)sulfonyl]amino}pentanamide (28).

(Alternate name: 5-Bromo-4-methoxyphenylsulfonylamido-Arg-Phe-NH₂).

This compound was synthesized as described in Method 1, except that 5-bromo-4-methoxyphenylsulfonyl chloride (571.10 mg, 2 mmol) was used in place of 1-naphthalenesulfonyl chloride.

Data: ESMS 569(MH+); [a]_{D}=+7.9 (c=1% W/V in methanol);

Compound 1026

N1-[(1S)-2-Amino-1-benzyl-2-oxoethyl]-(2S)-{[amino(imino)methyl]amino}-2-[2-nitrophenyl)sulfonyl]amino}pentanamide (29).

(Alternate name: 2-Nitrophenylsulfonylamido-Arg-Phe-NH₂).

This compound was synthesized as described in Method 1, except that 2-nitrophenylsulfonyl chloride (443.24 mg, 2 mmol) was used in place of 1-naphthalenesulfonyl chloride.

Data: ESMS 506 (MH+); [a]_{D}=−38.1 (c=1% W/V in methanol)

Compound 1027

N1-[(1S)-2-Amino-1-benzyl-2-oxoethyl]-(2S)-{[amino(imino)methyl]amino}-2-[2-cyanophenyl)sulfonyl]amino}pentanamide (30).

(Alternate name: 2-Cyanophenylsulfonylamido-Arg-Phe-NH₂).

This compound was synthesized as described in Method 1, except that 2-cyanophenylsulfonyl chloride (403.26 mg, 2 mmol) was used in place of 1-naphthalenesulfonyl chloride.

Data: ESMS 486(MH+); ¹H NMR (CD₃OD) d 1.6 (m, b, 4H), 2.75 (dd, J=4.4, 9.5 Hz, 1H), 3.00 (dd, J=5.3, 5.2 Hz, 1H), 3.12 (m, 2H), 3.9(m, 1H), 4.32 (m, 1H), 7.25 (m, 5H), 7.62 (m, 1H), 7.9 (m 1H);

Compound 1028

N1-[(1S)-2-Amino-1-benzyl-2-oxoethyl]-(2R)-{[amino(imino)methyl]amino}-2-[4-fluorophenyl)sulfonyl]amino}pentanamide (31).

(Alternate name: 4-Fluorophenylsulfonylamido-(D)Arg-Phe-NH₂).

This compound was synthesized as described in Method 1, except that (D)Arginine(Pbf) was used in place of (L)Arginine(Pbf), and 4-fluorophenylsulfonyl chloride (389.22 mg, 2 mmol) was used in place of 1-naphthalenesulfonyl chloride.

Data: ESMS 479(MH+);

Compound 1029

N1-[(1R)-2-Amino-1-benzyl-2-oxoethyl]-(2S)-{[amino(imino)methyl]amino}-2-[2-naphthalene)sulfonyl]amino}pentanamide (32).

(Alternate name: 2-Naphthalenesulfonylamido-Arg-(D)Phe-NH₂).

This compound was synthesized as described in Method 1, except that (D)Phenylalanine was used in place of (L)Phenylalanine, and 2-naphthalenesulfonyl chloride (453.36 mg, 2 mmol) was used in place of 1-naphthalenesulfonyl chloride.

Data: ESMS 510(MH+);

Compound 1030

N1-[(1S)-2-Amino-1-benzyl-2-oxoethyl]-(2R)-{[amino(imino)methyl]amino}-2-[2-bromophenyl)sulfonyl]amino}pentanamide (33).

(Alternate name: 2-Bromophenylsulfonylamido-(D)Arg-Phe-NH₂).

This compound was synthesized as described in Method 1, except that (D)Arginine(Pbf) was used to substitute (L)Arginine(Pbf), and 2-bromophenylsulfonyl chloride (511.04 mg, 2 mmol) was used in place of 1-naphthalenesulfonyl chloride.

Data: ESMS 540(MH+);

Compound 3001

N1-[(1S)-2-Amino-1-benzyl-2-oxoethyl]-(2R)-{[amino(imino)methyl]amino}-2-[1-naphthalene)sulfonyl]amino}pentanamide (34).

(Alternate name: 1-Naphthalenesulfonylamido-(D)Arg-Phe-NH₂).

This compound was synthesized as described in Method 1, except that (D)Arginine(Pbf) was used in place of (L)Arginine(Pbf).

Data: ESMS 511 (MH+)

Compound 1031

N1-[(1R)-2-Amino-1-benzyl-2-oxoethyl]-(2S)-{[amino(imino)methyl]amino}-2-[2-bromophenyl)sulfonyl]amino}pentanamide (35).

(Alternate name: 2-Bromophenylsulfonylamido-Arg-(D)Phe-NH₂).

This compound was synthesized as described in Method 1, except that (D)Phenylalanine was used to substitute (L)Phenylalanine, and 2-bromophenylsulfonyl chloride (511.04 mg, 2 mmol) was used in place of 1-naphthalenesulfonyl chloride.

Data: ESMS 540(MH+);

Compound 1032

N1-[(1R)-2-Amino-1-benzyl-2-oxoethyl]-(2S)-{[amino(imino)methyl]amino}-2-[2,6-difluorophenyl)sulfonyl]amino}pentanamide (36).

(Alternate name: 2,6-Difluorophenylsulfonylamido-Arg-(D)Phe-NH₂).

This compound was synthesized as described in Method 1, except that (D)Phenylalanine was used to substitute (L)Phenylalanine, and 2,6-difluorophenylsulfonyl chloride (425.20 mg, 2 mmol) was used in place of 1-naphthalenesulfonyl chloride.

Data: ESMS 511(MH+);

Compound 1033

N1-[(1R)-2-Amino-1-benzyl-2-oxoethyl]-(2S)-{[amino(imino)methyl]amino}-2-[4-fluorophenyl)sulfonyl]amino}pentanamide (37).

(Alternate name: 4-Fluorophenylsulfonylamido-Arg-(D)Phe-NH₂).

This compound was synthesized as described in Method 1, except that (D)Phenylalanine was used to substitute (L)Phenylalanine, and 4-fluorophenylsulfonyl chloride (389.22 mg, 2 mmol) was used in place of 1-naphthalenesulfonyl chloride.

Table 1. Summary of Compounds Tested.

TABLE 1

| | | Amino Acid |
|---|---|---|
| Compound | R-group | Chirality |
| 1001 | 1-naphthalene- | Both (L) |
| 1002 | 3-nitrobenzene- | Both (L) |
| 1003 | 4-nitrobenzene- | Both (L) |
| 1004 | 2,6-difluorobenzene- | Both (L) |
| 1005 | 4-fluorobenzene- | Both (L) |
| 1006 | 4-chlorobenzene- | Both (L) |
| 2001 | 2-bromobenzene- | Both (L) |
| 1007 | p-tolyl- | Both (L) |
| 1008 | phenyl- | Both (L) |
| 1009 | 4-methoxybenzene- | Both (L) |
| 1010 | 2,4-dichlorobenzene- | Both (L) |
| 1011 | α-toluene- | Both (L) |
| 1012 | 4-iodobenzene- | Both (L) |
| 1013 | 2-thiophene- | Both (L) |
| 1014 | 2-naphthalene | Both (L) |
| 1015 | 3,4-dimethoxybenzene- | Both (L) |
| 1016 | 4-chloro-3- | Both (L) |
| | nitrobenzene | |
| 2002 | 2,4-dinitrobenzene- | Both (L) |
| 1017 | 3-chloro-4- | Both (L) |
| | fluorobenzene- | |
| 1018 | 2-nitro-4- | Both (L) |
| | trifluoromethylbenzene- | |
| 1019 | 2,6-dichlorobenzene | Both (L) |
| 1020 | 3-(2,5- | Both (L) |
| | dichlorothiophene)- | |
| 2003 | 2-methoxy-4- | Both (L) |
| | methylbenzene- | |
| 1021 | 2,5-dichlorobenzene | Both (L) |
| 1022 | 3,4-dichlorobenzene | Both (L) |
| 1023 | 3-cyanobenzene- | Both (L) |
| 1024 | pentafluorobenzene- | Both (L) |
| 1025 | 5-bromo-2- | Both (L) |
| | methoxybenzene- | |
| 1026 | 2-nitrobenzene- | Both (L) |

| | | Amino Acid |
|---|---|---|
| Compound | R-group | Chirality |
| 1027 | 2-cyanobenzene- | Both (L) |
| 1028 | 4-fluorophenyl- | (D)Arg, (L)Phe |
| 1029 | 2-naphthalene- | (L)Arg, (D)Phe |
| 1030 | 2-bromophenyl- | (D)Arg, (L)Phe |
| 3001 | 1-naphthalene- | (D)Arg, (L)Phe |
| 1031 | 2-bromophenyl- | (L)Arg, (D)Phe |
| 1032 | 2,6-difluorophenyl- | (L)Arg, (D)Phe |
| 1033 | 4-fluorophenyl- | (L)Arg, (D)Phe |

II. Testing of Chemical Compounds.

Test 1—Radioligand Binding Assays

The binding properties of the compounds of the present invention were evaluated at cloned NPFF receptors using protocols described in PCT International Publication No. WO 00/18438, the disclosure of which is hereby incorporated by reference in its entirety into this application.

Test 2

Compounds were tested at concentrations ranging from 0.001 nM to 3600 nM, unless otherwise noted. EC₅₀ values and the corresponding intrinsic activities (I.A.) are given as percentages of a maximal response by NPFF. The data shown are representative of at least two independent experiments.

Table 2. Binding and Functional Activities of a Key Series of compounds Toward Rat NPFF Receptor Subtypes.

The binding data reflect competitive displacement of ([¹²⁵I]1DMeNPFF).

TABLE 2

| | Ki (nM) | | Functional Activity | | | |
|---|---|---|---|---|---|---|
| | Values | | rNPFF-1 | rNPFF-1 | rNPFF-2 | rNPFF-2 |
| Compound | rNPFF-1 | rNPFF-2 | EC₅₀ (nM) | I.A. % | EC₅₀ (nM) | I.A. % |
| 1001 | 261 | 1447 | 38 | 88 | 527 | 81 |
| 1002 | 136 | 1254 | 139 | 88 | 846 | 89 |
| 1003 | 732 | 2609 | 149 | 74 | 1871 | 44 |
| 1004 | 173 | 1447 | 117 | 79 | >3160 | 43 |
| 1005 | 150 | 1366 | 104 | 71 | 3496 | 46 |
| 1006 | 266 | 1014 | 151 | 75 | 3725 | 43 |
| 2001 | 112 | 2982 | 679 | 81 | >3160 | 09 |
| 1007 | 756 | 3083 | 286 | 79 | 2295 | 55 |
| 1008 | 321 | 4409 | 4698 | 70 | 5621 | 20 |
| 1009 | 321 | 1086 | Nd | Nd | Nd | Nd |
| 1010 | 871 | 1862 | 594 | 85 | 2980 | 28 |
| 1011 | 5959 | >10000 | 765 | 74 | 1342 | 62 |
| 1012 | 1427 | 2920 | 358 | 71 | 1418 | 93 |
| 1013 | 211 | 6393 | 135 | 80 | >3160 | 42 |
| 1014 | 314 | 2784 | 52 | 74 | 906 | 73 |
| 1015 | 462 | >10000 | 140 | 84 | 1815 | 74 |
| 1016 | 151 | 2090 | 62 | 72 | 660 | 81 |
| 2002 | 1387 | 5489 | 3160 | 34 | >10000 | 05 |
| 1017 | 1136 | 3564 | 376 | 84 | >3160 | 45 |
| 1018 | 1949 | 4430 | >3160 | 21 | 5621 | 10 |
| 1019 | 815 | 3375 | 2196 | 56 | >3160 | 45 |
| 1020 | 1954 | 5152 | >10000 | 01 | >10000 | 02 |
| 2003 | 2181 | >10000 | 461 | 102 | >3160 | 52 |
| 1021 | 335 | 2031 | 1027 | 78 | 2330 | 59 |
| 1022 | Nd | Nd | 1863 | 86 | >3160 | 34 |
| 1023 | 496 | 9919 | 166 | 90 | >3160 | 28 |
| 1024 | 486 | 5396 | 720 | 69 | >3160 | 43 |
| 1025 | 328 | 4122 | 596 | 78 | >3160 | 33 |
| 1026 | 535 | 3498 | 412 | 79 | >3160 | 59 |
| 1027 | 515 | 6171 | 183 | 52 | >3160 | 48 |

Table 3. Binding and Functional Activities of D-Arg- or D-Phe-Containing Compounds Toward Rat NPFF Receptor Subtypes.

In this series, one or both of the Arginine or Phenylalanine residues are changed to their corresponding D-isomer. This modification is expected to further improve the stability of these compounds against enzymatic degradation.

TABLE 3

| | Ki (nM) | | Functional Activity | | | |
|---|---|---|---|---|---|---|
| | Values | | rNPFF-1 | rNPFF-1 | rNPFF-2 | rNPFF-2 |
| Compound | rNPFF-1 | rNPFF-2 | EC₅₀ (nM) | I.A. % | EC₅₀ (nM) | I.A. % |
| 1028 | 1285 | 8056 | 404 | 46 | 1583 | 62 |
| 1029 | 399 | 2689 | 477 | 30 | >3160 | 86 |
| 1030 | 251 | 6200 | 655 | 35 | 1641 | 71 |
| 3001 | 46 | 2863 | >10000 | 1 | 378 | 79 |
| 1031 | 2574 | 6029 | 856 | 32 | 1574 | 24 |
| 1032 | 1289 | >10000 | 644 | 47 | 2758 | 61 |
| 1033 | 458 | >10000 | 1597 | 42 | 1941 | 57 |

Test 3

The effects of compounds on the micturition reflex were assessed in the “distension-induced rhythmic contraction” (DIRC) model (also called “volume-induced rhythmic contraction” model) in rats, as described in previous publications (e.g. Maggi, et al., 1987; Morikawa, et al., 1992; Guarneri, et al., 1993). This model is widely considered to be predictive for the actions of drugs to treat human urge incontinence (also referred to as detrusor instability or unstable bladder). Examples of drugs that are active in this model which also are used therapeutically in humans include oxybutynin and baclofen (Morikawa et al, 1992); imipramine and nortriptyline (36); and nifedipine and terodiline (37).

DIRC Model

Female Sprague Dawley rats weighing approximately 300 g were anesthetized with subcutaneous urethane (1.29/kg). The trachea was cannulated with PE240 tubing to provide a clear airway throughout the experiment. A midline abdominal incision was made and the left and right ureters were isolated. The ureters were ligated distally (to prevent escape of fluids from the bladder) and cannulated proximally with PE10 tubing. The incision was closed using 4-0 silk sutures, leaving the PE10 lines routed to the exterior for the elimination of urine. The bladder was canulated via the transurethral route using PE50 tubing inserted 2.5 cm beyond the urethral opening. This cannula was secured to the tail using tape and connected to a pressure transducer. To prevent leakage from the bladder, the cannula was tied tightly to the exterior urethral opening using 4-0 silk.

To initiate the micturition reflex, the bladder was first emptied by applying pressure to the lower abdomen, and then filled with normal saline in 100 μL increments (maximum=2 ml) until spontaneous bladder contractions occurred (typically 20-40 mmHg) at a rate of one contraction every 2 to 3 minutes. Once a regular rhythm was established, vehicle (saline) or test compounds were administered i.v. to examine their effects on bladder activity. The effect of a compound which inhibited the micturition reflex was expressed as its “disappearance time”, defined as the time between successive bladder contractions in the presence of the test compound minus the time between contractions before compound administration.

DIRC Model

Female Sprague Dawley rats weighing approximately 300 g were anesthetized with subcutaneous urethane (1.2 g/kg). The trachea was cannulated with PE240 tubing to provide a clear airway throughout the experiment. A midline abdominal incision was made and the left and right ureters were isolated. The ureters were ligated distally (to prevent escape of fluids from the bladder) and cannulated proximally with PE10 tubing. The incision was closed using 4-0 silk sutures, leaving the PE10 lines routed to the exterior for the elimination of urine. The bladder was canulated via the transurethral route using PE50 tubing inserted 2.5 cm beyond the urethral opening. This cannula was secured to the tail using tape and connected to a pressure transducer. To prevent leakage from the bladder, the cannula was tied tightly to the exterior urethral opening using 4-0 silk.

To initiate the micturition reflex, the bladder was first emptied by applying pressure to the lower abdomen, and then filled with normal saline in 100 μL increments (maximum=2 ml) until spontaneous bladder contractions occurred (typically 20-40 mmHg) at a rate of one contraction every 2 to 3 minutes. Once a regular rhythm was established, vehicle (saline) or test compounds were administered i.v. to examine their effects on bladder activity. The effect of a compound which inhibited the micturition reflex was expressed as its “disappearance time”, defined as the time between successive bladder contractions in the presence of the test compound minus the time between contractions before compound administration.

Results of Test 3

N-(6-chloro-4-methyl-2-quinolinyl)guanidine at a dose of 1 mg/kg, i.v. produced complete inhibition of distention induced contractions of the rat bladder, resulting in a disappearance time of 35 minutes. N-(4,7-dimethyl-2-quinolinyl)guanidine at a dose of 3 mg/kg, i.v. produced complete inhibition of distention induced contractions of the rat bladder, resulting in a disappearance time of 12 minutes.

Discussion of Test 3

These results represent the first demonstration that synthetic ligands which are active as agonists at the NPFF2 receptor inhibit the micturition reflex. In this regard their actions mimic the action of the endogenous peptide ligand NPFF. The ability of these compounds to inhibit the micturition reflex in this model can be taken as an indication that they will be effective in the treatment of urge incontinence in humans (see above).

Discussion

The compounds discussed above can be classified as agonists and antagonists based on the following parameters: an agonist has an intrinsic activity (IA)>15%, while an antagonist has a Ki≦1.2 μM and an intrinsic activity (IA)≦15% at the rat cloned neuropeptide FF (NPFF) receptors.

Based on this definition the compounds can be classified as follows:

Compounds 1001 to 1033 are concurrently agonists at NPFF1 and NPFF2 receptors;

Compounds 2001 to 2003 are agonists at NPFF1 receptors, with weak or no affinity to NPFF2 receptors; and

Compound 3001 is concurrently an antagonist at NPFF1 receptors and an agonist at NPFF2 receptors.

When tested in the assays described and used in Tests 1 and 2, the known compound dansyl-RFamide has the following properties: Ki at rNPFF-1=88 nM, Ki at rNPFF-2=2310 nM, EC50 at rNPFF-1=524 nM (I.A. 106%), EC 50 at rNPFF-2=2524 nM (I.A. 96%). Thus, compounds described and claimed herein can be selected based on Table 2 and 3 which have enhanced potency at one or both of the tested receptors relative to a known compound, or compounds which have the unexpected property of being an antagonist at both of the tested receptors.

### REFERENCES

1. Yang, H. Y., Fratta, W., Majane, E. A., and Costa, E. Isolation, sequencing, synthesis, and pharmacological characterization of two brain neuropeptides that modulate the action of morphine. Proc. Natl. Acad. Sci. U.S.A. 82(22):7757-7761, 1985.

2. Vilim, E. S., Ziff, E. Cloning of the neuropeptide NPFF and NPAF precursor form bovine, rat, mouse, and human. Soc. Neurosci. 21:760, 1995.

3. Bonini, J. A., Jones, K. A., Adham, N., Forray, C., Artymyshyn, R., Durkin, M. M., Smith, K. E., Tamm, J. A., Boteju, L. W., Lakhlani, P. P., Raddatz, R., Yao, W-J., Ogozaleck, K. L., Boyle, N., Kouranova, E. V., Quan, Y., Vyase, P. J., Wetzel, J. M., Branchek, T. A., Gerald, C., Borowsky, B. Identification and characterization of two G protein-coupled receptors for neuropeptide FF. J. Biol. Chem. 275(50): 39324-31, 2000.

4. DNA Encoding Mammalian Neuropeptide FF (NPFF) Receptors and Uses Thereof. WO 00/18438.

5. Panula, P., Aarnisalo, A. A., and Wasowicz, K. Neuropeptide FF, a mammalian neuropeptide with multiple functions [published erratum appears in Prog. Neurobiol. 1996 June; 49(3):285]. Prog. Neurobiol. 48(4-5):461-487, 1996.

6. Roumy, M. and Zajac, J. M. Neuropeptide FF, pain and analgesia. Eur. J. Pharmacol. 345(1):1-11, 1998.

7. Payza, K., Akar, C. A., and Yang, H. Y. Neuropeptide FF receptors: structure-activity relationship and effect of morphine. J. Pharmacol. Exp. Ther. 267(1):88-94, 1993.

8. Raffa, R. B., Kim, A., Rice, K. C., de Costa, B. R., Codd, E. E., and Rothman, R. B. Low affinity of FMRFamide and four FaRPs (FMRFamide-related peptides), including the mammalian-derived FaRPs F-8-Famide (NPFF) and A-18-Famide, for opioid mu, delta, kappa 1, kappa 2a, or kappa 2b receptors. Peptides 15(3):401-404, 1994.

9. Malin, D. H., Lake, J. R., Arcangeli, K. R., Deshotel, K. D., Hausam, D. D., Witherspoon, W. E., Carter, V. A., Yang, H. Y., Pal, B., and Burgess, K. Subcutaneous injection of an analog of neuropeptide FF precipitates morphine abstinence syndrome. Life Sci. 53(17):PL261-6, 1993.

10. Malin, D. H., Lake, J. R., Fowler, D. E., Hammond, M. V., Brown, S. L., Leyva, J. E., Prasco, P. E., and Dougherty, T. M. FMRF-NH2-like mammalian peptide precipitates opiate-withdrawal syndrome in the rat. Peptides 11(2):277-280, 1990.

11. Panula, P., Kivipelto, L., Nieminen, O., Majane, E. A., and Yang, H. Y. Neuroanatomy of morphine-modulating peptides. Med. Biol. 65(2-3):127-135, 1987.

12. Allard, M., Geoffre, S., Legendre, P., Vincent, J. D., and Simonnet, G. Characterization of rat spinal cord receptors to FLFQPQRFamide, a mammalian morphine modulating peptide: a binding study. Brain Res. 500(1-2):169-176, 1989.

13. Allard, M., Zajac, J. M., and Simonnet, G. Autoradiographic distribution of receptors to FLFQPQRFamide, a morphine-modulating peptide, in rat central nervous system. Neuroscience 49(1):101-116, 1992.

14. Gouarderes, C., Tafani, J. A. M., and Zajac, J. M. Affinity of neuropeptide FF analogs to opioid receptors in the rat spinal cord. Peptides 19(4):727-730, 1998.

15. Payza, K. and Yang, H. Y. Modulation of neuropeptide FF receptors by guanine nucleotides and cations in membranes of rat brain and spinal cord. J. Neurochem. 60(5):1894-1899, 1993.

16. Gouarderes, C., Sutak, M., Zajac, J. M., and Jhamandas, K. Antinociceptive effects of intrathecally administered F8Famide and FMRFamide in the rat. Eur. J. Pharmacol. 237(1):73-81, 1993.

17. Yang, H. Y. T., Martin, B. M. Soc. Neurosci. 21, 760, 1995.

18. Jhamandas, K., Sutak, M., Yang, H. -Y. T. Soc. Neurosci. 22, 1313, 1996.

19. Kontinen, V. K., Aarnisalo, A. A., Idaenpaeaen-Heikkilae, J. J., Panula, P., and Kalso, E. Neuropeptide FF in the rat spinal cord during carrageenan inflammation. Peptides 18(2):287-292, 1997.

20. Wei, H., Panula, P., and Pertovaara, A. A differential modulation of allodynia, hyperalgesia and nociception by neuropeptide FF in the periaqueductal gray of neuropathic rats: Interactions with morphine and naloxone. Neuroscience 86(1):311-319, 1998.

21. Oberling, P., Stinus, L., Le Moal, M., and Simonnet, G. Biphasic effect on nociception and antiopiate activity of the neuropeptide FF (FLFQPQRFamide) in the rat. Peptides 14(5):919-924, 1993.

22. Gouarderes, C., Jhamandas, K., Sutak, M., and Zajac, J. M. Role of opioid receptors in the spinal antinociceptive effects of neuropeptide FF analogues. Br. J. Pharmacol. 117(3):493-501, 1996.

23. Gouarderes, C., Kar, S., Zajac, J. -M., Neuroscience, 74, 21-27, 1996.

24. Xu, M.; Kontinen, V. K.; Panula, P.; Kalso, E. Peptides, 10, 1071-1077, 1999.

25. Coudoré, M. A.; Courteix, C.; Eschalier, A.; Zajac, J. -M., Wilcox, G. L.; Fialip, J. Resumes de la lere Reunion de la Societe Francaise de Pharmacologie (Marseilles, France), vol. 17, p23, 1997.

26. Huang, E. Y. -K.; Li, J. Y.; Tan, P. P. -C.; Wong, C. -H.; Chen, J. -C. Peptides, 21, 205-210, 2000.

27. Robert, J. J., Orosco, M., Rouch, C., Jacquot, C., and Cohen, Y. Unexpected responses of the obese “cafeteria” rat to the peptide FMRF-amide. Pharmacol. Biochem. Behav. 34(2):341-344, 1989.

28. Murase, T., Arima, H., Kondo, K., and Oiso, Y. Neuropeptide FF reduces food intake in rats. Peptides 17(2):353-354, 1996.

29. Kavaliers, M., Hirst, M., and Mathers, A. Inhibitory influences of FMRFamide on morphine- and deprivation-induced feeding. Neuroendocrinology. 40(6):533-535, 1985.

30. Payza, K. and Yang, H. Y. Modulation of neuropeptide FF receptors by guanine nucleotides and cations in membranes of rat brain and spinal cord. J. Neurochem. 60(5):1894-1899, 1993.

31. Devillers, J. P., Mazarguil, H., Allard, M., Dickenson, A. H., Zajac, J. M., and Simonnet, G. Characterization of a potent agonist for NPFF receptors: binding study on rat spinal cord membranes. Neuropharmacology 33(5):661-669, 1994.

32. Allard, M., Labrouche, S., Nosjean, A., and Laguzzi, R. Mechanisms underlying the cardiovascular responses to peripheral administration of NPFF in the rat. J. Pharmacol. Exp. Ther. 274(1):577-583, 1995.

33. Laguzzi, R., Nosjean, A., Mazarquil, H., and Allard, M. Cardiovascular effects induced by the stimulation of neuropeptide FF receptors in the dorsal vagal complex: An autoradiographic and pharmacological study in the rat. Brain Res. 711(1-2):193-202, 1996.

34. Brussard, A. B.; Kits, A.; Ter Maat, A. H.; Mulder, A. H. and Schoffelmeer, A. N. M., Peptides 10, 735, 1989.

35. Bourguignon, J. J.; Collot, V.; Didier, B.; Laulin, J. P. and Simmonet, G. Analogs of NPFF, a neuropeptide which modulates morphine analgesia: Proceedings of the XIVth International Symposium on Medicinal Chemistry, Awouters, F. (Ed.), 1997, Elsevier Science B. V., pp 35-44.

36. Pietra, C., Poggesi, E., Angelico, P., Guarneri, L. and Testa, R. Effects of some antidepressants on the volume-induced reflex contractions of the rat urinary bladder: lack of correlation with muscarinic receptors affinity. Pharmacological Research, 22: 421-432, 1990.

37. Guarneri, L., Ibba, M., Angelico, P., Colombo, D., Fredella, B. and Testa, R. Effects of drugs used in the therapy of detrusor hyperactivity on the volume-induced contractions of the rat urinary bladder. Pharmacological Research, 27: 173-187, 1993.

38. Morgan, D. G., Small, C. J., Abusnana, S., Turton, M., Gunn, I., Heath, M., Rossi, M., Goldstone, A. P., O'Shea, D., Meeran, K., Ghatei, M., Smith, D. M., and Bloom, S. The NPY Y1 receptor antagonist BIBP 3226 blocks NPY induced feeding via a non-specific mechanism. Regul. Pept. 75-76: 377-382, 1998.

39. Maggi, C. A, Furio, M., Santicioli, P., Conte, B. and Meli, A. (1987) “Spinal and supraspinal components of GABAergic inhibition of the micturition reflex in rats. J. Pharmacol Exp Ther 240, 998-1005.

40. Morikawa, K., Hashimoto, S., Yamauchi, T., Kato, H., Ito, Y., and Gomi, Y. (1992) “Inhibitory effect of inaperisone hydrochloride (inaperisone), a new centrally acting muscle relaxant, on the micturition reflex” Eur J pharmacol 213, 409-415.

What is claimed is:

1. A compound having the structure: 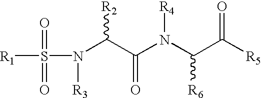
wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,
wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,
wherein Y is O or S;
wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;
wherein R2 is —(CH₂)ₙ—NH—C(═NH)—NH₂; —(CH₂)n-C(═NH)—NH₂; —(CH₂)ₙ—N(R7)₂; or -J;
wherein n is an integer between 1 and 6, and wherein J is any of the following structures:
wherein each of R3 and R4 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; or C5-C7 cycloalkenyl; 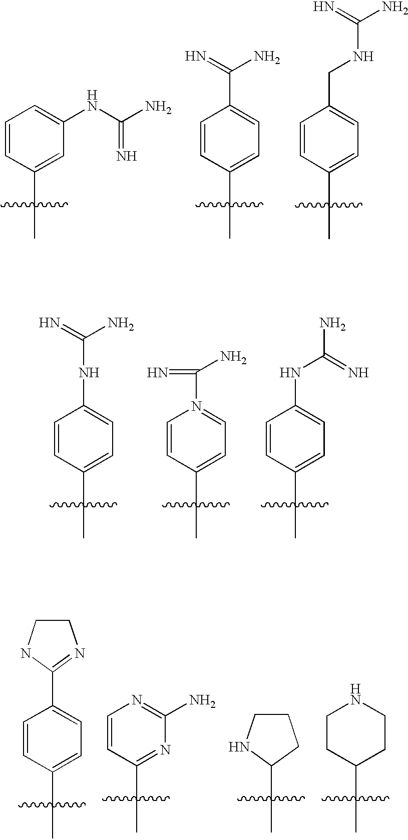
wherein R5 is —OR8 or —N(R8)₂;
wherein each R8 is independently H, straight chained or branched C1-C7 alkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;
wherein R6 is arylalkyl, heteroarylalkyl, aryl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,
or a pharmaceutically acceptable salt thereof.

2. The compound of claim 1, having the structure: 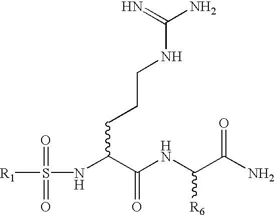
wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,
wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═O—Y)R7, —C(═Y)OR7, —N(R7)₂, —C(═Y)NR7, —NR7C(═Y)R7 or —N(R7)C(═Y)N(R7)₂,
wherein Y is O or S;
wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; and
wherein R6 is arylalkyl, heteroarylalkyl, aryl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,
or a pharmaceutically acceptable salt thereof.

3. The compound of claim 1, having the structure: 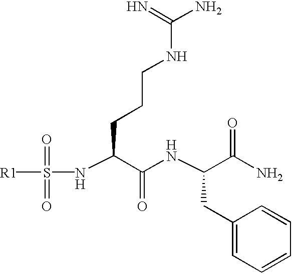
wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; or arylalkyl, heteroarylalkyl, phanyl or heteroaryl,
wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂₁—C(═Y)NR7, —NR7C(═Y)R7 or —N(R7)C(═Y)N(R7)₂.

4. The compound of claim 1, wherein the compound has the structure:

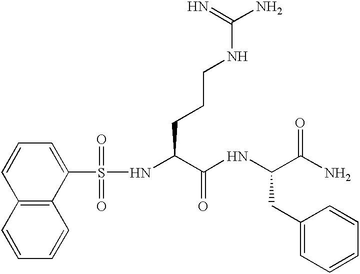

5. The compound of claim 1, wherein the compound has the structure:

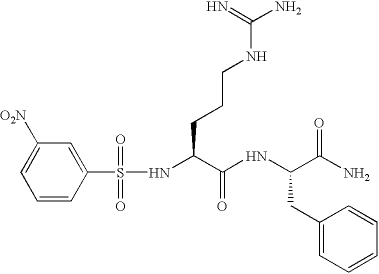

6. The compound of claim 1, wherein the compound has the structure:

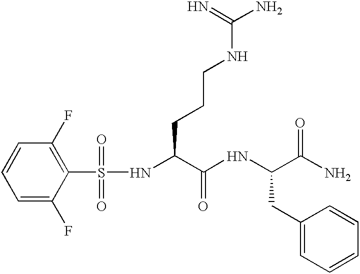

7. The compound of claim 1, wherein the compound has the structure:

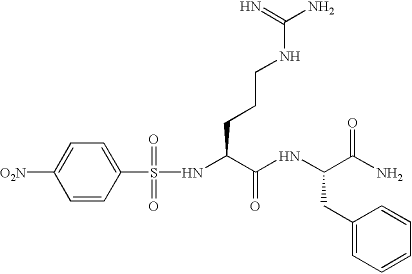

8. The compound of claim 1, wherein the compound has the structure:

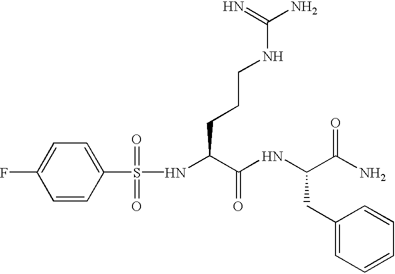

9. The compound of claim 1, wherein the compound has the structure:

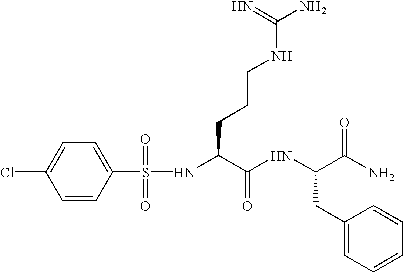

10. The compound of claim 1, wherein the compound has the structure:

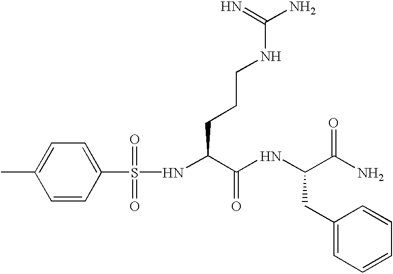

11. The compound of claim 1, wherein the compound has the structure:

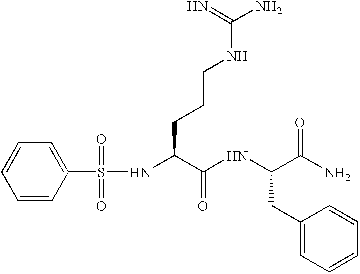

12. The compound of claim 1, wherein the compound has the structure:

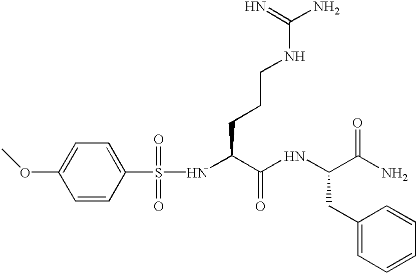

13. The compound of claim 1, wherein the compound has the structure:

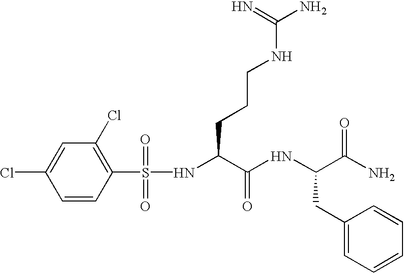

14. The compound of claim 1, wherein the compound has the structure:

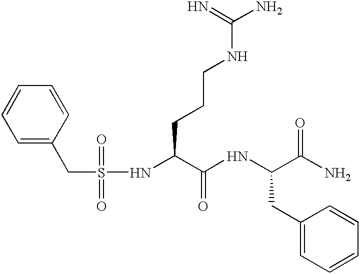

15. The compound of claim 1, wherein the compound has the structure:

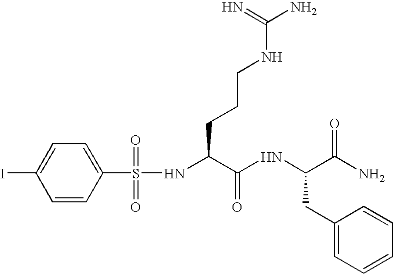

16. The compound of claim 1, wherein the compound has the structure:

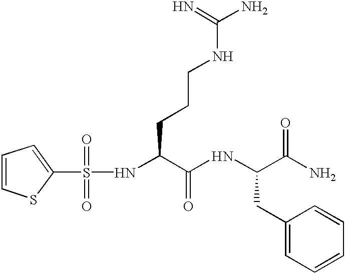

17. The compound of claim 1, wherein the compound has the structure:

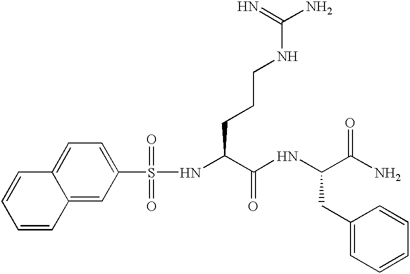

18. The compound of claim 1, wherein the compound has the structure:

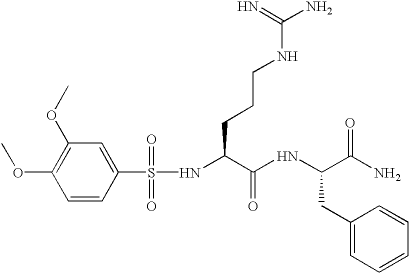

19. The compound of claim 1, wherein the compound has the structure:

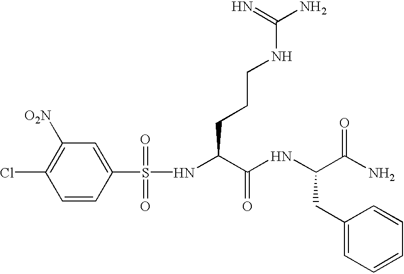

20. The compound of claim 1, wherein the compound has the structure:

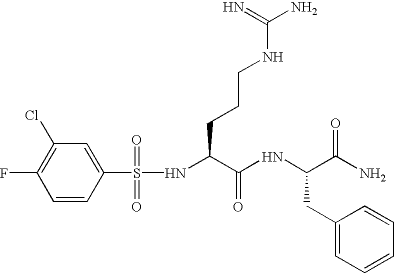

21. The compound of claim 1, wherein the compound has the structure:

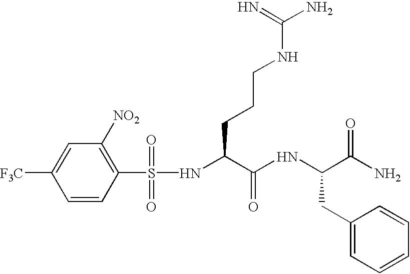

22. The compound of claim 1, wherein the compound has the structure:

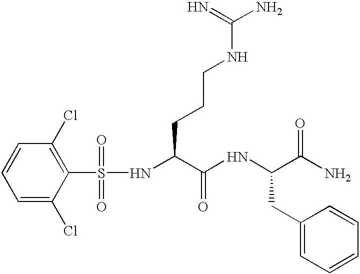

23. The compound of claim 1, wherein the compound has the structure:

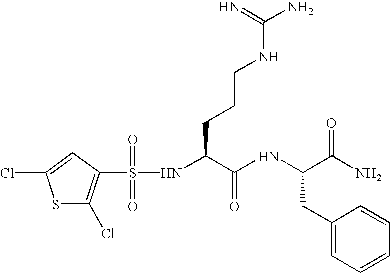

24. The compound of claim 1, wherein the compound has the structure:

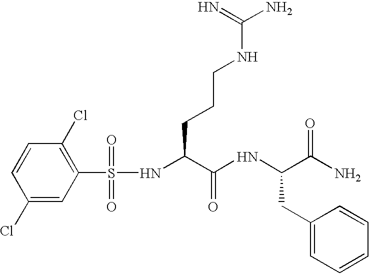

25. The compound of claim 1, wherein the compound has the structure:

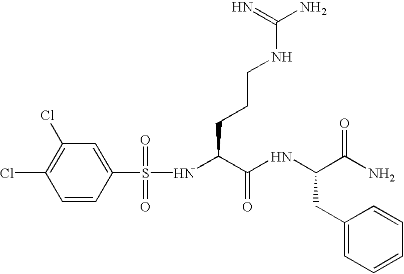

26. The compound of claim 1, wherein the compound has the structure:

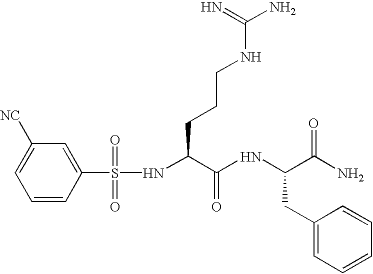

27. The compound of claim 1, wherein the compound has the structure:

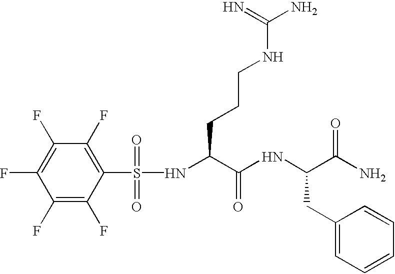

28. The compound of claim 1, wherein the compound has the structure:

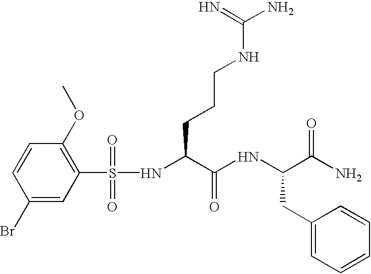

29. The compound of claim 1, wherein the compound has the structure:

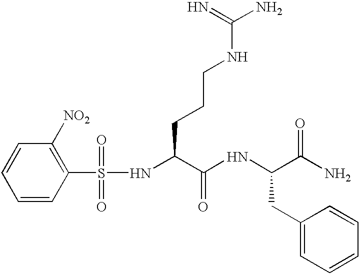

30. The compound of claim 1, wherein the compound has the structure:

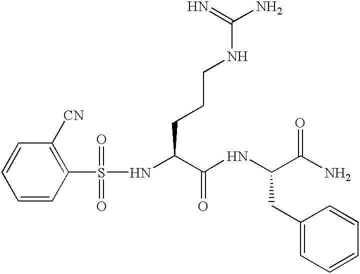

31. The compound of claim 1, wherein the compound has the structure:

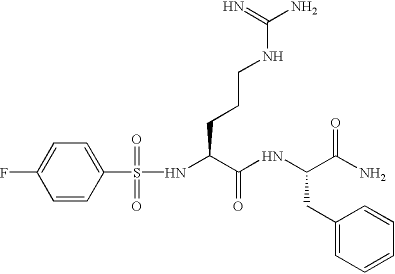

32. The compound of claim 1, wherein the compound has the structure:

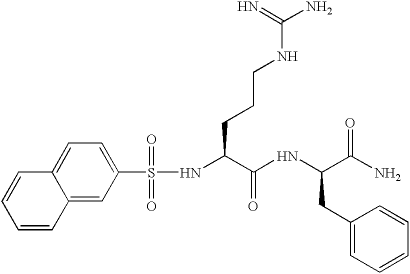

33. The compound of claim 1, wherein the compound has the structure:

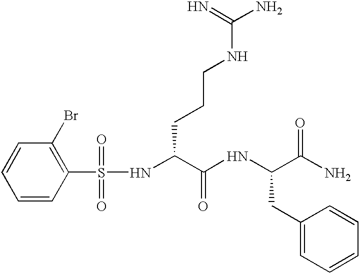

34. The compound of claim 1, wherein the compound has the structure:

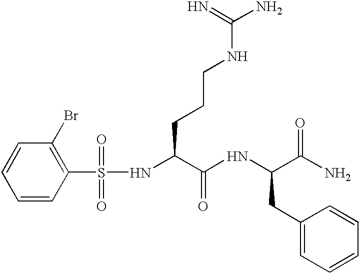

35. The compound of claim 1, wherein the compound has the structure:

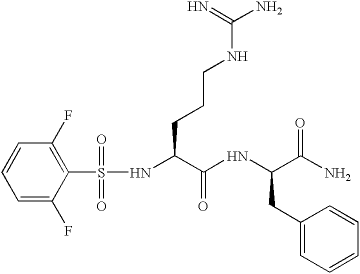

36. The compound of claim 1, wherein the compound has the structure:

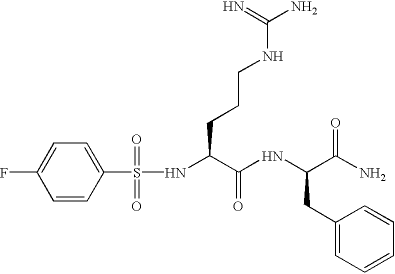

37. The compound of claim 1, wherein the compound has the structure:

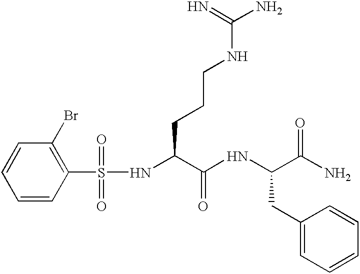

38. The compound of claim 1, wherein the compound has the structure:

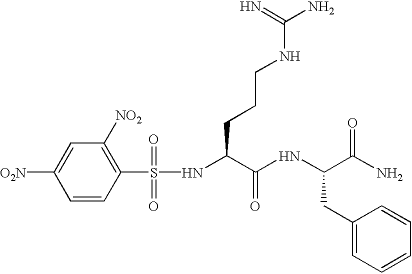

39. The compound of claim 1, wherein the compound has the structure:

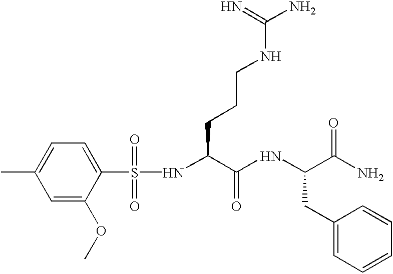

40. The compound of claim 1, wherein the compound has the structure:

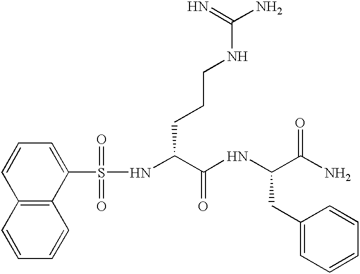

41. A pharmaceutical composition comprising the compound of any one of claims 1-39 and a pharmaceutically acceptable carrier.

42. The pharmaceutical composition of claim 41, wherein the carrier is phosphate buffered saline, physiological saline or water.

43. A method of preparing a pharmaceutical composition comprising mixing the compound of any one of claims 1-40 with a pharmaceutical acceptable carrier.

44. The method of claim 43, wherein the carrier is phosphate buffered saline, physiological saline or water.

45. A compound which is converted in vivo to the compound of any one of claims 1-40.

46. A compound which is a metabolite of the compound of any one of claims 1-40.

47. A salt of the compound of any one of claims 1-40.

48. A method of treating pain in a subject in need of such treatment comprising administering to the subject an effective amount of a compound having the structure: 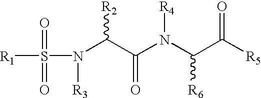
wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,
wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,
wherein Y is O or S;
wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;
wherein R2 is —(CH₂)ₙ—NH—C(═NH)—NH₂; —(CH₂)n—C(═NH)—NH₂; —(CH₂)ₙ—N(R7)₂; or -J;
wherein n is an integer between 1 and 6, and wherein J is any of the following structures: 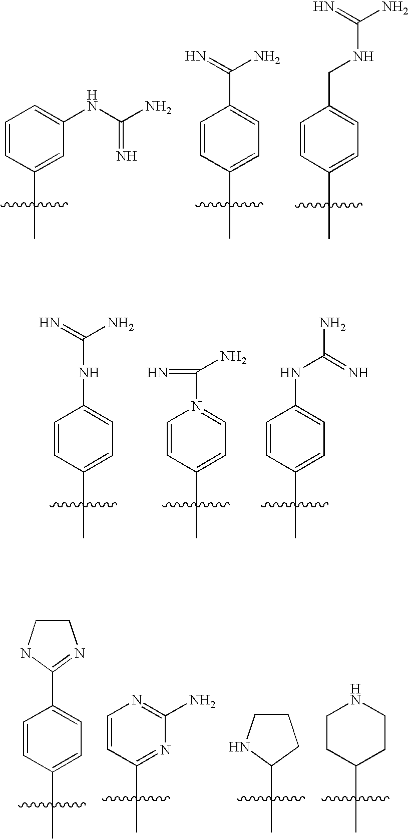
wherein each of R3 and R4 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; or C5-C7 cycloalkenyl;
wherein R5 is —OR8 or —N(R8)₂;
wherein each R8 is independently H, straight chained or branched C1-C7 alkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, polyfluorocycloalkyl; or C5-C7 cycloalkenyl;
wherein R6 is arylalkyl, heteroarylalkyl, aryl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂,
or a pharmaceutically acceptable salt thereof,
to thus treat the pain in the subject.

49. The method of claim 48, wherein the compound has the structure: 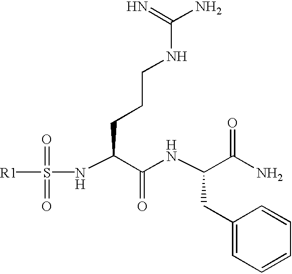
wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,
wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂.

50. A method of treating a lower urinary tract disorder in a subject in need of such treatment comprising administering to the subject an effective amount of a compound having the structure: 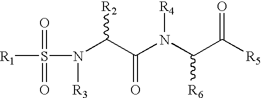
wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,
wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂₁—C(═Y)NR7, —NR7C(═Y)R7 or —N(R7)C(═Y)N(R7)₂,
wherein Y is O or S;
wherein R7 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;
wherein R2 is —(CH₂)ₙ—NH—C(═NH)—NH₂; —(CH₂)n—C(═NH)—NH₂; —(CH₂)ₙ—N(R7)₂; or J;
wherein n is an integer between 1 and 6, and wherein J is any of the following structures: 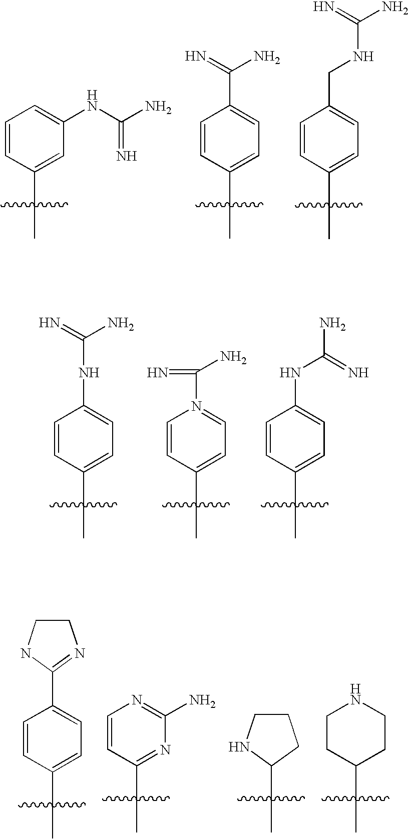
wherein each of R3 and R4 is independently H, straight chained or branched C1-C7 alkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; or C5-C7 cycloalkenyl;
wherein R5 is —OR8 or —N(R8)₂;
wherein each R8 is independently H, straight chained or branched C1-C7 alkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl;
wherein R6 is naphthyl, arylalkyl, heteroarylalkyl, phenyl or heteroaryl, each optionally substituted with one or more of the following substituents: halogen, hydroxy, C1-C6 alkoxy, aryloxy, straight chained or branched C1-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂—C(═Y)NR7, —NR7C(═Y)R7 or —N(R7)C(═Y)N(R7)₂,
or a pharmaceutically acceptable salt thereof,
to thus treat the lower urinary tract disorder in the subject.

51. The method of claim 50, wherein the compound has the structure: 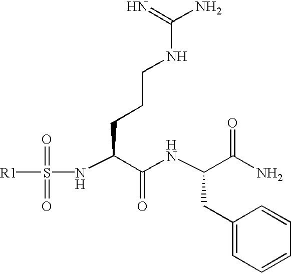
wherein R1 is straight chained or branched C1-C7 alkyl, hydroxyalkyl, alkoxyalkyl, alkylthioalkyl, monofluoroalkyl or polyfluoroalkyl; straight chained or branched C2-C7 alkenyl or alkynyl; C3-C7 cycloalkyl, monofluorocycloalkyl, or polyfluorocycloalkyl; C5-C7 cycloalkenyl; naphthyl; arylalkyl, heteroarylalkyl, phenyl or heteroaryl,
wherein the arylalkyl, heteroarylalkyl, phenyl or heteroaryl is unsubstituted or substituted with one or more of the following substituents: halogen, hydroxy, C1-C6 alkoxy, aryloxy, straight chained or branched C2-C6 alkyl, aryl, heteroaryl, nitro, cyano, C1-C6 alkylthio, substituted or unsubstituted arylalkyl or heteroarylalkyl, —C(═Y)R7, —C(═Y)OR7, —N(R7)₂, —C(═Y)NR7, —NR7C(═Y)R7 or —N(R7)C(═Y)N(R7)₂.

52. The method of claim 50, wherein the lower urinary tract disorder is interstitial cystitis, stress incontinence or urge incontinence.

53. The method of claim 50, wherein the compound has the structure:

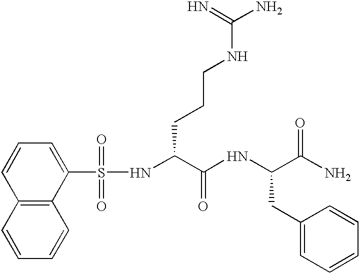

54. The method of claim 50, wherein the compound has the structure:

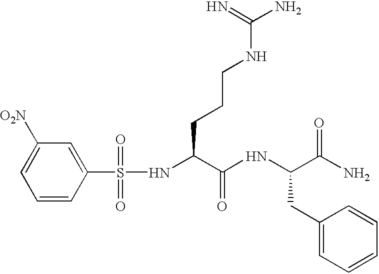

55. The method of claim 50, wherein the compound has the structure:

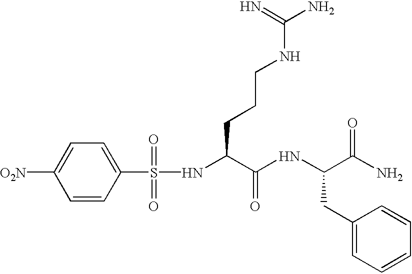

56. The method of claim 50, wherein the compound has the structure:

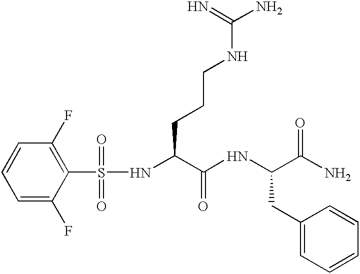

57. The method of claim 50, wherein the compound has the structure:

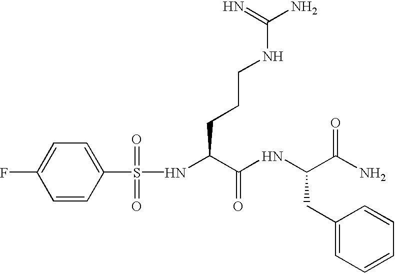

58. The method of claim 50, wherein the compound has the structure:

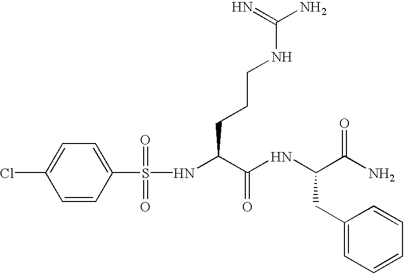

59. The method of claim 50, wherein the compound has the

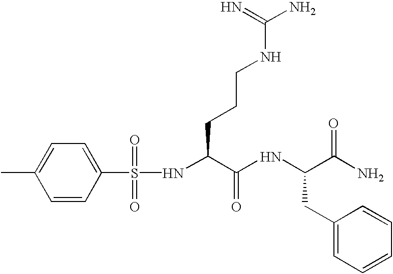

60. The method of claim 50, wherein the compound has the

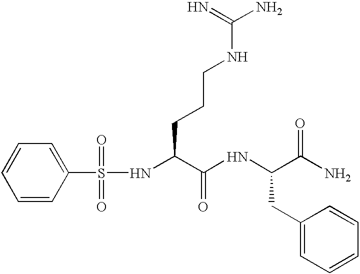

61. The method of claim 50, wherein the compound has the structure:

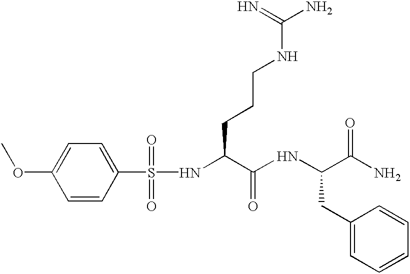

62. The method of claim 50, wherein the compound has the structure:

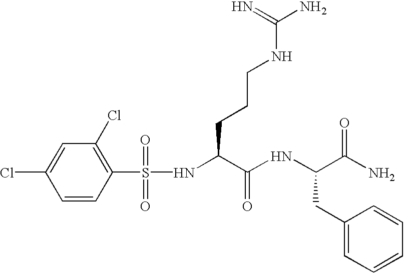

63. The method of claim 50, wherein the compound has the structure:

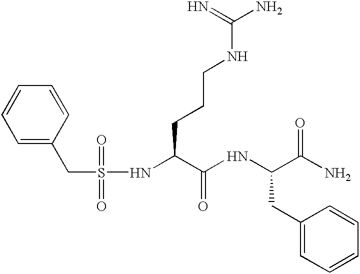

64. The method of claim 50, wherein the compound has the structure:

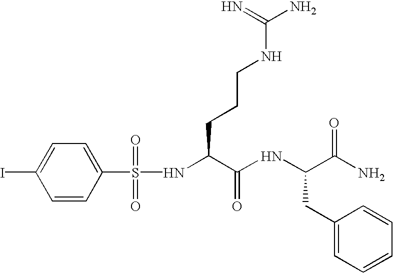

65. The method of claim 50, wherein the compound has the structure:

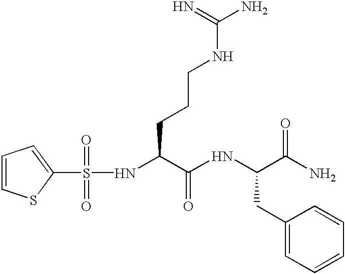

66. The method of claim 50, wherein the compound has the structure:

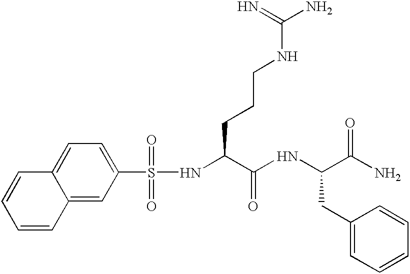

67. The method of claim 50, wherein the compound has the structure:

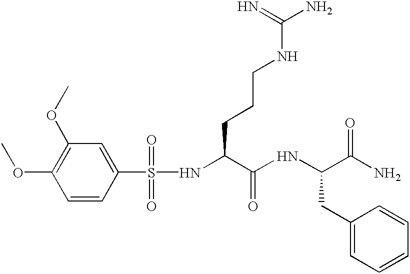

68. The method of claim 50, wherein the compound has the structure:

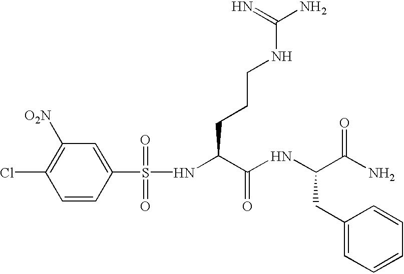

69. The method of claim 50, wherein the compound has the structure:

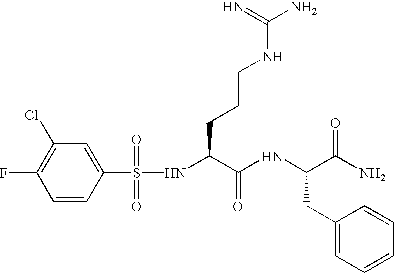

70. The method of claim 50, wherein the compound has the structure:

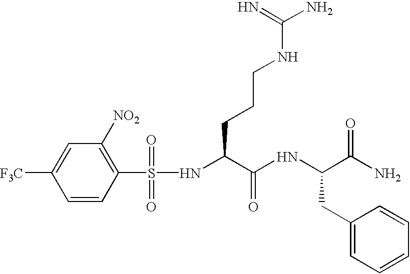

71. The method of claim 50, wherein the compound has the structure:

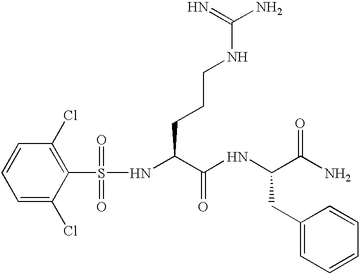

72. The method of claim 50, wherein the compound has the structure:

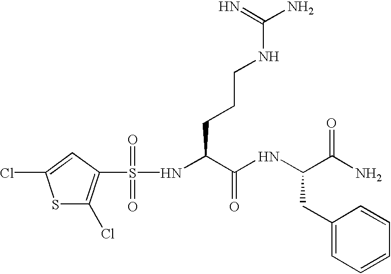

73. The method of claim 50, wherein the compound has the structure:

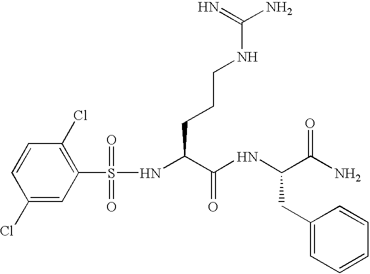

74. The method of claim 50, wherein the compound has the structure:

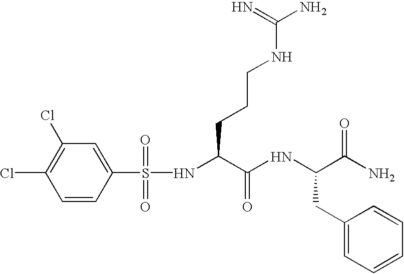

75. The method of claim 50, wherein the compound has the structure:

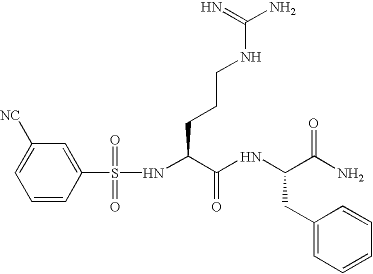

76. The method of claim 50, wherein the compound has the structure:

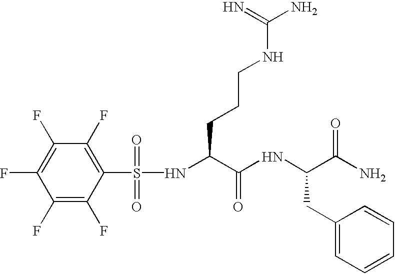

77. The method of claim 50, wherein the compound has the structure:

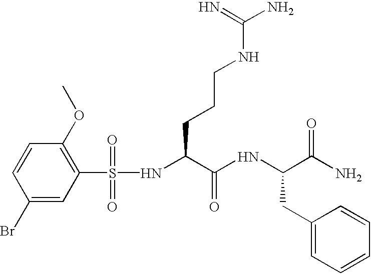

78. The method of claim 50, wherein the compound has the structure:

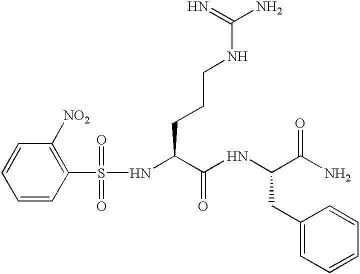

79. The method of claim 50, wherein the compound has the structure:

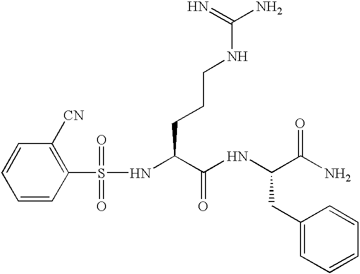

80. The method of claim 50, wherein the compound has the structure:

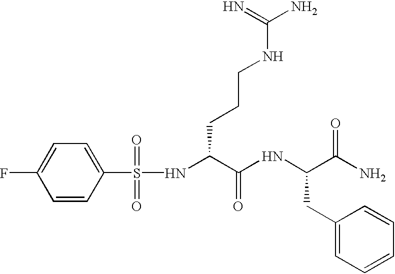

81. The method of claim 50, wherein the compound has the structure:

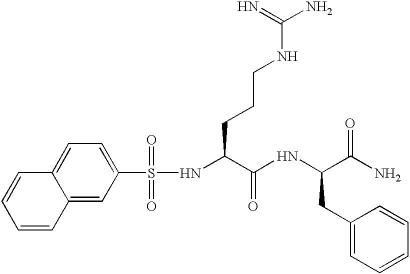

82. The method of claim 50, wherein the compound has the structure:

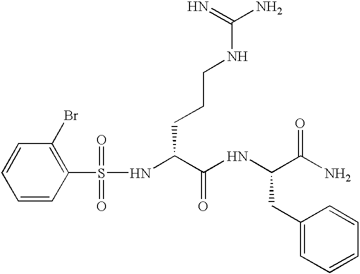

83. The method of claim 50, wherein the compound has the structure:

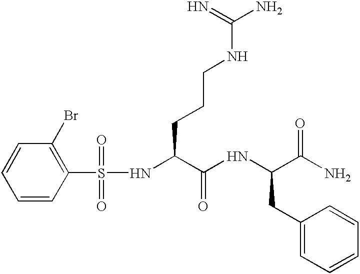

84. The method of claim 50, wherein the compound has the structure:

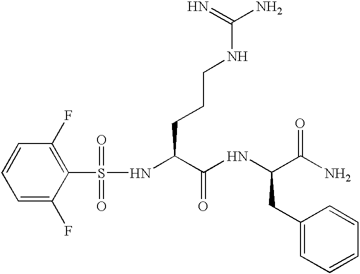

85. The method of claim 50, wherein the compound has the structure:

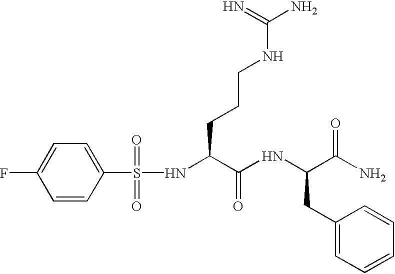

86. The method of claim 50, wherein the compound has the structure:

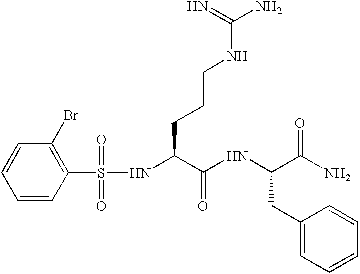

87. The method of claim 50, wherein the compound has the structure:

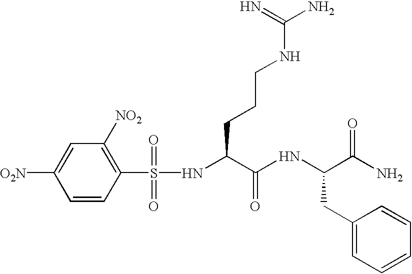

88. The method of claim 50, wherein the compound has the structure:

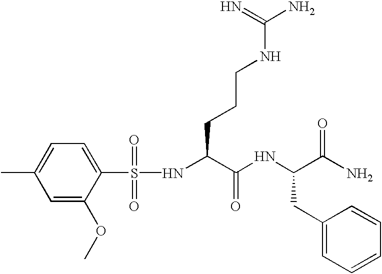

89. The method of claim 50, wherein the compound has the structure: [chemical structure]
